(19) United States
(12) Patent Application Publication
(10) Pub. No.: US 2014/0268163 A1
(43) Pub. Date: Sep. 18, 2014

(54) Methods and Apparatus Related to Multi Wavelength Photothermal Optical Coherence Tomography

(71) Applicants: Thomas E. Milner, Austin, TX (US); Roman Kuranov, Carey, NC (US); Timothy Duong, San Antonio, TX (US)

(72) Inventors: Thomas E. Milner, Austin, TX (US); Roman Kuranov, Carey, NC (US); Timothy Duong, San Antonio, TX (US)

(21) Appl. No.: 13/990,595

(22) PCT Filed: Nov. 30, 2011
(86) PCT No.: PCT/US11/62617

§ 371 Date: May 16, 2014

Related U.S. Application Data
(60) Provisional application No. 61/418,300, filed on Nov. 30, 2010.

(51) Int. Cl. A61B 6/03; G01N 21/31

(52) U.S. Cl. CPC A61B 6/03; G01N 21/31
USPC 356/451

(57) ABSTRACT

Embodiments of the invention include apparatus and methods of non-invasively detecting one or more constituents of a target using multiple wavelength photothermal optical coherence tomography.

### STATEMENT REGARDING PRIORITY CLAIM

This application claims priority to U.S. Provisional Patent Application Ser. No. 61/418,300 filed Nov. 30, 2010, which is incorporated by reference in its entirety.

### STATEMENT REGARDING FEDERALLY FUNDED RESEARCH

This invention was made with government support under PID 130610, Fund 41016, Dept. R4700: 2010 IIMS Mentored Research Career Development (KL2) Scholar in Clinical and Translational Science: “Depth-Resolved Functional Optical Coherence Tomography of Diabetic Retinopathy.” Effective August 2010 awarded by the University of Texas Health Science Center at San Antonio. The government has certain rights in the invention.

### I. FIELD OF THE INVENTION

Field of the invention is generally related to physics, biology, medicine and imaging. In certain embodiments the field of the invention is directed to multiple wavelength photothermal optical coherence tomography.

### II. BACKGROUND

Optical Coherence Tomography (OCT) is a high-resolution optical tomography technique using an optical interference phenomenon. This technique is capable of realizing a high resolution (approximately 1 to 10 μm), close to the optical wavelength, by employing the optical interference phenomenon. Furthermore, a probe used to capture a tomographic image is an optical probe, and therefore X-ray exposure does not pose a problem, in contrast to X-ray CT (Computed Tomography). Using these high resolution and non-invasive qualities, diagnosis apparatuses for observing the posterior of the eye and the anterior eye portion at a high resolution on a par with a microscope is realized through OCT. Various OCT methods exist, namely time domain OCT (TD-OCT) and frequency domain OCT (FD-OCT).

Non-invasive analysis is a valuable technique for acquiring information about systems or targets without undesirable side effects, such as damaging or contaminating the system being analyzed. In the case of analyzing living entities, such as human tissue, undesirable side effects of invasive analysis include the risk of infection along with pain and discomfort associated with the invasive process. In measuring components in the blood, it is highly desirable to measure the blood level frequently and accurately to provide appropriate treatment. A non-invasive method would avoid the pain and risk of infection and provide an opportunity for frequent or continuous measurement.

Non-invasive analysis based on several techniques have been proposed. These techniques include: near infrared spectroscopy using both transmission and reflectance; spatially resolved diffuse reflectance; frequency domain reflectance; fluorescence spectroscopy; polarimetry and Raman spectroscopy. These techniques are vulnerable to inaccuracies due to issues such as, environmental changes, presence of varying amounts of interfering contamination, skin heterogeneity and variation of location of analysis. These techniques also require considerable processing to de-convolve the required measurement, typically using multi-variate analysis and have typically produced insufficient accuracy and reliability for an intended application.

There is a need for additional methods and apparatus for commercially viable, robust, non-invasive devices with ability to measure concentrations of various components in a target, such as, but not limited to human tissue.

### SUMMARY

Embodiments of the invention include apparatus, systems, and methods for non-invasively detecting one or more constituents of a target. The target can be a biological or non-biological target. The detection methods expose a target comprising one or more constituents to one or more excitation radiation produced by an excitation radiation source. The excitation radiation source produces 1, 2, 3, 4 or more excitation wavelengths that are differentially absorbed by one or more constituents in the target. In certain aspects the excitation radiation is electromagnetic radiation. Electromagnetic radiation (EMR) is a form of energy exhibiting wave like behavior as it travels through space. EMR has both electric and magnetic field components, which oscillate perpendicular to each other and perpendicular to the direction of energy propagation. EMR is classified according to the frequency of its oscillations. In order of increasing frequency and decreasing wavelength, these include radio waves, microwaves, infrared radiation, visible light, ultraviolet radiation, X-rays and gamma rays. In certain aspects the excitation radiation has a wavelength in the radio wave frequency (wavelength of about 10⁸ m) to ultraviolet frequency (a wavelength of about 10⁻⁸ m). EMR carries energy and momentum that may be imparted to matter with which it interacts. The excitation radiation can be absorbed by a constituent of a target and converted into heat or another detectable manifestation. In certain embodiments the amplitude, frequency and/or phase of the EMR can be modulated or coded.

In certain aspects a constituent is a molecular entity that is at least temporarily within or associated with a target that is exposed to the excitation radiation. In a further aspect a target may have one or more constituents having variant forms that differentially absorb the excitation radiation.

Non-invasive methods for detecting constituents in a sample generally comprise excitation of a target constituent(s) with an excitation radiation source, detection of physical changes in and around the target constituent(s) using phase sensitive optical coherence tomography (OCT) or other interferometric technique, and processing the data collected using phase sensitive OCT.

Certain embodiments are directed to methods for measuring concentration of a constituent or the relative concentration of a first constituent respective to a second constituent in a target comprising: (a) exposing a target having 1, 2, 3, 4 or more constituents to a first excitation radiation at a first wavelength that is absorbed by at least a first constituent and, if a second constituent is targeted, a second excitation radiation at a second wavelength that is absorbed by at least a second constituent, (b) measuring optical path length changes of light returning from the target resulting from exposure of the target to at least a first and optionally a second excitation radiation, and (c) determining a difference between the changes in optical path length (i) prior to, during and/or after exposure to an excitation radiation or (ii) relative to the first and the second excitation radiation and determining the levels of the first constituent relative to the second constituent by evaluating the optical path length changes. In certain aspects the relative concentration of two constituents is determined, e.g., concentration of a first or second constituent divided by the sum of the first and second constituent. In certain aspects the first excitation radiation is differentially absorbed by the first and second constituent. In certain aspects one wavelength of excitation radiation is selected that is within about 1, 5, 10, 15 or 20 nm, μm, mm, or m of an isobestic point of two constituents. In spectroscopy, an isobestic point is a specific wavelength at which two chemical species have the same molar absorptivity (ε). When an isobestic plot is constructed by the superposition of the absorption spectra of two species (whether by using molar absorptivity for the representation, or by using absorbance and keeping the same molar concentration for both species), the isobestic point corresponds to a wavelength at which these spectra cross each other. In still a further aspect optical path length changes are determined at 2, 3, 4, 5, 6, 7, 8, 9, or 10 or more different depths within the target. In other aspects the optical path length changes are determined at a plurality of points in one or more planes of a target, e.g., such an array of path length differences can be used to form a topographical representation of constituent across 1, 2, or 3 dimensions of a target. Data or measurements from a plurality of points can be acquired by scanning or simultaneously by using wide beam exposure and 1D or 2D array of detectors. In certain aspects the target is exposed to at least a first and second excitation radiation at the same time. The target can be exposed to a plurality wavelengths, thus 2, 3, 4, 5, 6, 7, 8, 9, 10 or more different excitation wavelengths can be used. The target can be exposed to the plurality of wavelengths simultaneously or generally time or frequency encoded.

Certain embodiments are directed to methods for mapping the presence of one or more constituent in a target comprising: (a) exposing a target having at least one constituent to at least a first excitation radiation at least a first wavelength that is absorbed by at least a first constituent, (b) measuring optical path length changes in the target resulting from exposure of the target to at least the first excitation radiation at a plurality of locations within and/or on the target, and (c) mapping the changes in optical path length to at least the first excitation radiation at the plurality of location within and/or on the target. In certain aspects 1, 2, 3, 4 or more constituents are exposed to an excitation radiation that is differentially absorbed by a constituent in the target. The excitation radiation wavelength is selected so that the pathlength changes can be attributed to a particular constituent. In certain aspect 1, 2, 3, 4, or more constituents are mapped within or on a target.

In certain aspects measuring optical path length changes is by phase sensitive optical coherence tomography.

The target can be a biological or non-biological target. The biological target can be, but is not limited to a tissue, organ, or biological fluid. In certain aspects a target is in a subject. In further aspects the biological target is a portion of an organ or tissue, such as the retina, choroid, skin, tumor, epithelia, blood vessel, cervix, prostate, stomach, large intestine, small intestine, esophagus, tongue, mouth, or brain. In a further aspect the biological target is the retina or a blood vessel in the retina.

In certain aspects a target is an endogenous (e.g, a biomolecule such as a metabolite or protein) or exogenous (e.g., nanoparticle or other contrast or dye agent) molecule, compound, or composition. In certain aspects the target comprises a composition, compound, molecule, or biomolecule that absorbs energy at least at the first wavelength. In a further aspect the biomolecule is a protein, a nucleic acid, a carbohydrate, a lipid, a metabolite, or a combination thereof. In certain aspects the biological target comprises hemoglobin and/or variants of hemoglobin. In a further aspect the first constituent is oxygenated hemoglobin. In still a further aspect the second constituent is deoxygenated hemoglobin, carboxy-hemoglobin, sulf-hemoglobin, or methemo-hemoglobin.

Certain embodiments include methods for measuring hemoglobin oxygen saturation (SaO₂). In certain aspects the first excitation radiation has a wavelength of about 790 nm to about 820 nm. In a further aspect the first excitation radiation has a wavelength of about 800 nm. In certain aspects the second excitation radiation has a wavelength of about 750 nm to about 775 nm or about 840 nm to about 900 nm. In a further aspect the second excitation radiation has a wavelength of about 765 nm.

In certain embodiments the target is a non-biological target. The non-biological target can be a pharmaceutical composition, a film, a polymeric composition or a material, gas, or solution that comprises one or more constituents that differentially absorb at two or more wavelengths of radiation and can be monitored using phase sensitive OCT.

Certain embodiments include a single or multi wavelength photothermal optical coherence tomography apparatus comprising (a) an excitation radiation source configured to produce excitation radiation, in certain embodiments the excitation radiation source is capable of producing at least one or two distinct excitation wavelengths, (b) a phase sensitive optical coherence detector, and (c) data processing unit configured to process data related to optical path length changes in a target exposed to an excitation radiation.

Certain aspects provide a system for providing assessment of a target by multi wavelength photothermal optical coherence tomography, the system comprising (a) an application server comprising (i) an input manager to receive data from a phase sensitive optical coherence detector configured to obtain data related to photothermal effects in a target, and (ii) a data processor to provide assessment of the difference in optical properties of a target using at least one or two different wavelengths of excitation obtained from the target; and (b) a network server comprising an output manager constructed and arranged to provide a target assessment, one or more constituent concentration, and/or an image to a user.

Further aspects provide a computer readable medium having software modules for performing a method comprising the acts of (a) comparing differences in optical characteristics of a target resulting from excitation at 1, 2, 3, 4, or more different wavelengths; and (b) providing an assessment of optical properties or relative difference in optical properties for use in determining an image, and/or a relative quantity of a constituent in the target.

Still further aspects provide a computer system, having a processor, memory, external data storage, input/output mechanisms, a display, for assessing a target by single or multi wavelength photothermal optical coherence tomography comprising (a) logic mechanisms in the computer for processing optical data obtained from a target exposed to an excitation radiation; and (c) an analysis method run by the computer for assessing optical properties or comparing the differences in optical properties resulting from excitation with at least 1, 2, 3, 4, or more excitation wavelengths and determining the status of at least one constituent in a target.

Certain embodiments are directed to methods of measuring the consumption or production of a constituent by a target comprising measuring flow of a fluid comprising one or more constituent to a target, measuring flow of the fluid comprising the one or more constituents from a target, and measuring the level of one or more constituents in the fluid flowing to the target and the fluid flowing from the target; and determining alteration, consumption, or production of one or more constituents by the target using the difference in levels of one or more constituents in the fluid flowing to a target relative to levels of one or more constituents in a fluid flowing from a target. In certain aspects the flow to a target, flow from a target, and levels of one or more constituent flowing to and from a target are measured in 0.01, 0.1, 10, or 20 millisecond or seconds of each other, including all values and ranges there between. In certain aspects the measurements are with 20 milliseconds or less. In certain aspects the flow to a target, flow from a target, and levels of one or more constituent flowing to and from a target are measured simultaneously. The target can be a biological or non-biological target. The flow to and from a target can be measured using Doppler OCT or using other flow determining apparatus. The constituent levels can be measured using a photothermal OCT process and/or apparatus as described herein. Certain aspects the target is the retina. In further aspects, the method is used to assess retinal physiology in a subject having or suspected of having diabetes or diabetic retinopathy.

As used herein the term “excitation,” unless otherwise indicated, refers to the photothermal excitation produced by the absorbtion of radiation.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Multi Wavelength Photothermal-OCT. A tunable Ti:Al₂O₃ laser (765 and 800 nm) was utilized as an excitation source.

FIGS. 2A-2B. (A) M-mode phase map and (B) intensity OCT A-scan. The lines of constant phase in the M-mode image and spikes on the A-scan correspond to 1—the upper air-vessel interface (optical pathlength, op=73 μm), 2—upper vessel-blood interface (op=187 μm), 3—lower blood-vessel interface (op=572 μm), 4—vessel-epoxy interface (op=676 μm), 5—epoxy-glass slide interface (op=749 μm).

FIGS. 3A-3C. (A) Amplitude of optical pathlength (op) variation at depths 1-5 vs frequency, grey trace in FIG. 3A is magnitude of Fourier transform of FIG. 3C. (B) Typical op time profile at depth 5 (reference SaO₂=18.5%, excitation at 800 nm). Shutter was opened at 2 s and closed at 5.4 s (indicated by arrows). (C) is segment of FIG. 3B after subtracting the linear trend used for calculation of op amplitude at 42 Hz (indicated by vertical dashed lines in FIG. 3B).

FIG. 4 Absorption spectra of oxy- and deoxy-hemoglobin. Dashed lines indicate the laser excitation wavelengths (765 nm and 800 nm). Data compiled by Prahl et al.

FIG. 5 SaO₂ levels measured with DWP-OCT using 765 nm and 800 nm excitation light vs reference avoximeter readings. DWP-OCT SaO₂ levels were calculated from depths d5−d1 (diamonds) and d3−d₂ (squares).

FIG. 6 The sketch of the probe for DWP OCT and cuvette with blood sample. The thickness of the cuvette's walls are 50-200 μm, and the thickness of the cuvette's lumen is 80-300 μm. The distance between OCT probe and cuvette, thickness of the cuvette's walls and thickness of the cuvette's lumen are calibrated, possibly with Intensity OCT. The probe and excitation lights from the OCT probe are focused on the cuvette to provide sufficient intensity of the excitation light. The parameters of the excitation light is also calibrated so the intensity of the excitation light at the cuvette is known. 1 is a front air/cuvette interface, 2—front cuvette/blood interface, 3—bottom bloodcuvette interface, 4—bottom cuvette air interface. Each of the interfaces can be coated to increase the reflection of OCT light and not influence or slightly influence excitation light in a known manner for optimizing signal to noise ratio of the DWP OCT.

FIGS. 7A-7C. DWP-OCT for in vivo SaO₂ measurements (A), exposed brain cortex and the probe on top of an indicated by arrow 30-μm diameter arteriole (B) and M-mode OCT image recorded from a probed site (C). Optical pathlength changes (op) in a selected arteriole induced by photothermal excitation wavelengths 770 nm (op₁) and 800 nm (op₂) are measured by DWPOCT and converted to SaO₂ levels. Red spot under the probe (B) is specular reflection of photothermal excitation light from tissue. White spots on the fiber probe are photographic artifacts due to multiple reflections between the fiber and aluminum fiber holder. OCT-signal intensity A-scans of the M-mode image (C) calculated as a 20 Log of Fourier transformation of one laser sweep interference fringes between SMF-28 fiber end face and brain tissue. The green arrow (C) indicates position of the cover glass/cortex boundary, while the purple arrow indicates depth location of the arteriole (350 μm below the glass cortex boundary, 550 μm below fiber endface) extracted from speckle contrast and Doppler M-mode images (FIG. 8).

FIGS. 8A-8B. In-vivo murine brain M-mode image. Speckle contrast (A) and Doppler (B) OCT image. Speckle contrast and Doppler images are presented in color coded arbitrary units and radians correspondingly. Both images consist of 128 (time)×400 (depth) pixels. The 30-μm diameter target arteriole (purple arrow on left, 15-20 μm lumen diameter) at 550 μm optical depth is visualized in both speckle contrast and Doppler M-mode images. Three cardiac cycles are distinguished in the speckle contrast image. The maxima of the arteriole expansion in a cardiac cycle are indicated with orange arrows. Glass-tissue interface is at approximately 200 μm.

FIG. 9. DWP-OCT intensity (top) and phase (bottom) vs. time at fixed probe depth at the posterior side of a 30-μm diameter arteriole. Phase signal contains a fast (5.9 Hz) component due to heart beat and a slow (1.2 Hz) component due to respiration.

FIGS. 10A-10B. (A) Power spectrum of optical pathlength (op) variations at a fixed 350 μm tissue optical depth (550 μm from the fiber endface). The op power spectrum was acquired by converting time domain phase data presented at the FIG. 3 to op variation [(op(t)=φ(t)λ/(4π)] and using a Fast Fourier Transformation (FFT) of the first 5 s of the data. (B) Laser-induced op variations at 380 Hz (800 nm) and 400 Hz (770 nm) indicated by arrow in A.

FIG. 11. OP amplitude signal-to-noise ratio (op_{SNR}) vs. laser (800 nm) photothermal excitation frequency. Error bars are op_{SDR}±op_{SD}, n=5.

FIG. 12. Computed SaO₂ levels from DWP-OCT op measurement. Each data point is calculated from op measured over a 5 s time period at fixed probe depth. Successive data points are separated by 0.25 s. Solid lines indicate mean DWP-OCT values, dashed lines - means±standard deviations. Systemic SaO₂ levels measured with pulse oximeter indicated in red. Probe depth is on the posterior side of 30 μm diameter arterioles.

FIG. 13. DWP-OCT vs. pulse oximeter SaO₂ values. Average difference between DWP-OCT and pulse oximeter SaO₂ values is 10.1%. Error bars are standard deviations (n=21).

FIG. 14. Scheme of one of the realization of the two-arm functional OCT system for 2D and 3D imaging: 1 is a two arm sample interferometer and 2 is a laser excitation part. Excitation light is combined with OCT probe light via wavelength division multiplexor (WDM). BD is a balanced photodetector, Additional WDMs can be used to combine as many excitation wavelengths with OCT probe light as needed. M1, M2 are scanning mirrors.

FIG. 15. Scheme of the one of the realization of common-path functional OCT system for 2D and 3D imaging: 1 is an additional interferometer to set desired working distance of the signal interferometer (Subsystem 1) using delay line (DL). Excitation light can be combined with OCT probe light via WDMs in the same manner as in FIG. 14. M1,2 are scanning mirrors. FBG is a fiber Bragg grating in this specific realization.

FIGS. 16A-16B. Model of the microvessel distribution in the tissue used for calculation of the tissue area investigated during 5 s using functional OCT: view from the side (A) and from the front (B).

FIG. 17. Identification/tracking algorithm of the target microvessel during functional OCT measurements.

FIG. 18A-18B. In vivo (A), Intensity and (B) speckle fluctuation OCT images of the retina. Retinal (R) and choroid (Ch) blood vessels indicated with arrows. Shadows in choroid from large retinal blood vessels are indicated with the letters S.

FIG. 19. Amplitude of the op variation per 1 mW of photothermal excitation light with depth (orange) and its approximation with error function (blue): Erf(a[z−z₀]), where z₀=568 μm, a=0.031 l/μm (B). The estimated depth resolution of the DWP-OCT is 45 μm.

### DETAILED DESCRIPTION OF THE INVENTION

Optical coherence tomography (OCT) involves splitting broadband light into a probe and reference beam. The probe beam is applied to the system to be analyzed (the target). Light scattered back from the target is combined with the reference beam to form the measurement signal. Only light that is scattered from a depth within the target such that its total optical path length coincide with total optical path length of reference light within coherence length of source light are combined interferometrically. The interferometric signal provides a measurement of the scattering value at a plurality of depths within the target by modulating optical path length in the reference arm (time-domain OCT), or by analyzing the spectrum of interference fringes of broadband light (Fourier-Domain OCT). Thus the amount of light scattering back as a function of depth can be measured.

The photothermal effect is a phenomenon associated with electromagnetic radiation. It is produced by the photo-excitation of material, resulting in the production of thermal energy (heat). The “photothermal effect” is when electromagnetic radiation absorbed by one or more constituents is converted into thermal energy after the constituent is irradiated by a beam. Constituents and variants or isoforms of constituents differentially absorb light energy at various wavelengths (spatial period of the electromagnetic radiation) due to the molecular characteristics of the constituent(s)—this differential can be used in detecting and/or measuring various constituents in a target.

Certain aspects of the present invention use multiple wavelengths of electromagnetic radiation to photothermally excite one or more constituent in a target. An example of a multiple wavelength photothermal optical coherence tomography (MWP-OCT) set up is provided in FIG. 1. Such a system will typically contain: (1) excitation source(s) (e.g., laser(s)) and optional fiber delivery system; (2) a target to be evaluated; (3) Phase Sensitive (PhS) OCT system to measure target dependent optical path length changes induced by excitation laser light (Kuranov et al., 2010, which is incorporated herein by reference in its entirety); and (4) a processor to analyze data and generate one or more outputs.

A target of the system can include any material (solid, gas or liquid) that comprises one or more constituents that differentially absorb electromagnetic radiation at various wavelengths, resulting in a photothermal effect. The target can be a biological target, e.g., a tissue, organ, or body fluid, or a non-biological target containing one or more constituent or isoform thereof that differentially absorbs at one or more wavelengths relative to a second constituent.

A non-limiting example used to demonstrate this method is the non-invasive determination of oxygen saturation of hemoglobin using multiple wavelength photothermal (MWP) optical coherence tomography (OCT) based on the differential absorbtion of light by oxygenated hemoglobin and non-oxygenated hemoglobin. In the example case we used two-wavelength photothermal excitation thus this particular realization of method called Dual-Wavelength Photothermal (DWP) OCT. Specifically, when applied to the hemoglobin oxygen saturation levels (SaO₂) MWP and DWP OCT can be referenced as SaO₂-OCT. These methods can be used on various other biological and non-biological targets that comprise constituents that absorb electromagnetic radiation differentially. The inventors selected this example because the abnormal oxygenation of tissues and blood is an important biomarker indicating early progression of many diseases preceding irreversible tissue damage including cancer, inflammatory and infectious processes, diabetic retinopathy, choroidal disorders, stroke and vascular dementia among others (Carmeliet and Jain, 2000). The inventors note that this method can be used in a variety of biological and non-biological contexts.

Blood extraction for subsequent measurement of hemoglobin oxygen saturation (SaO₂) levels is invasive and can damage epithelial tissues. Moreover, during extraction highly oxygenated arterial blood can mix with less oxygenated venous blood in an unknown proportion introducing artifacts in measured SaO₂ values. Non-invasive methods to measure in vivo oxygen saturation (SaO₂) resolve problems associated with tissue damage and mixing artifacts. For example, difference in magnetic properties between oxy- and deoxy-hemoglobin underlies relative oxygenation level measurements using the blood oxygenation level-dependent (BOLD) MRI approach (Cheng et al., 2006). BOLD MRI can provide layer-specific relative oxygenation levels in the brain and ocular tissues but faces challenges for SaO₂ assessment due to poor temporal and spatial resolution.

Distinct differences in the absorption spectra between oxy- and deoxy-hemoglobin in the visible and infrared (IR) spectral regions underly previously reported spectroscopic methods for non-invasive assessment of in vivo hemoglobin oxygen saturation levels (Izhaky et al., 2009; Grinvald et al., 1986; Sirotin and Das, 2009; Jobsis, 1977; Reynolds et al., 1988; Matcher et al., 1995; Dunn et al., 2003; Grap, 2002; Kamat, 2002; Mythen, 2006). Because these spectroscopic methods provide mean arterial and venous (Izhaky et al., 2009; Grinvald et al., 1986; Sirotin and Das, 2009; Jobsis, 1977; Reynolds et al., 1988; Matcher et al., 1995; Dunn et al., 2003) and arterial (Grap, 2002; Kamat, 2002) SaO₂ averaged over a relatively large volume of tissue, spatial specificity to identify a damaged tissue volume is compromised. Assessment of in vivo SaO₂ levels with higher spatial specificity is required for early diagnosis and longitudinal monitoring of many diseases including cancer of epithelial tissues, local inflammatory and infectious processes, retinopathy, choroidal eye disorders and stroke. For example, the human retina is only 200-300 μm thick excluding the choroid and consists of many well defined physiological layers and has two independent vascular oxygen supplies (retina and choroid, which is about 400-600 μm). The choroidal vascular supply provides a ten-fold higher blood flow than the retinal supply and can introduce unpredictable artifacts when retinal SaO₂ levels are measured using scattering based spectroscopic methods. Spectral Fourier Domain Optical Coherence Tomography (FD OCT) (Leitgeb et al., 2000; Robles et al., 2009) can provide simultaneously high depth and temporal resolution and has been applied for assessing depth-resolved blood oxygenation levels (Faber et al., 2003; Kagemann et al., 2007; Lu et al., 2008; Yi and Li, 2010; Faber et al., 2005). Although SaO₂ measurements with Spectral OCT in the visible range (without an independent excitation source) was reported in phantoms where absorption of hemoglobin is maximal (Yi and Li, 2010), in the near infrared spectral region only a correlation with SaO₂ levels was shown (Lu et al., 2008; Faber et al., 2005). Spectral OCT in the visible range is limited to probing superficial tissue depths of a few hundred microns and is unsuitable to monitor ocular disorders due to high sensitivity of retinal photoreceptors and stimulation of the occipital lobe of the cerebral hemisphere.

Challenges to apply spectral OCT to measure SaO₂ levels are: (1) relatively low absorption of infrared light by oxy-deoxy-hemoglobin (4% energy absorption through 200 μm blood vessel at the isobestic 800 nm wavelength); (2) high variability in OCT signal amplitude due to speckle effects; and (3) trade-off between spectral and spatial resolutions (Robles et al., 2009; Robles et al., 2010). Instrumentation where phase changes in a sample are induced by absorption of single-wavelength excitation light and measured with phase sensitive (PhS) OCT (Zhao et al., 2000; Choma et al., 2005; Adler et al., 2007; Kuranov et al., 2010) are known as Photothermal OCT (Paranjape et al., 2010; Adler et al., 2008; Skala et al., 2008; Zhou et al., 2010). One example of the methods described herein is Dual-Wavelength Photothermal OCT (DWP-OCT) approach that uses excitation and probe light in the near infrared spectral region for layer-specific SaO₂ monitoring in tissues. Amplitude of DWP-OCT signal in response to absorption of excitation light is decoupled from the high variability of OCT signal intensity.

There is a need for precise measuring of the hemoglobin species in vitro from blood samples. Point-of-care instruments such as the Avoximeter family from ITC for measuring major hemoglobin species such as oxy-, deoxy-, carboxy-, and sulfhemoglobin providing critical life saving information and used intensively in cardiac labs and ICU units. The fastest, accurate and cost effective solution in whole blood cuvettes is provided by optical absorption based Avoximeter family instrumentation. Avoximeters rely on the differences in absorption spectra of the hemoglobin species. The main confounding factor in optical measurements of the concentrations of the distinct hemoglobin species is high scattering of the blood. The avoximeter family instrumentation basically optimize parameters of cuvettes (sample) and irradiation detection geometry to acquire most of the scattering light therefore minimizing errors due to unpredictable loss of the scattering light.

In the methods described herein the amplitude of the measured signal depends solely on absorption and does not depend on scattering. Therefore the main problem of measuring concentrations of the hemoglobin species is avoided. Additionally the thickness of the cuvette needed for precise measurements of the total hemoglobin concentration as opposed to the relative concentrations of the species in DWP OCT can be measured individually with high accuracy, thus eliminating the need of preliminary measurements of the cuvettes thickness reducing cost and increasing the precision of the approach.

The present methods provide cost effective measuring of two of the most important hemoglobin parameters: oxygen saturation levels (SaO₂) and total hemoglobin (THb). Furthermore, additional devices will be capable of measuring five major hemoglobin species including oxy-, deoxy-, carboxy-, and sulfhemoglobin.

### I. Measurement of Oxygen Saturation of Hemoglobin.

A “tissue phantom” or simply “phantom” is a synthetic control sample intended to mimic tissue when examined—one example of a blood vessel phantom is a PTFE tube containing blood. The ability of PTFE tubing to maintain a constant blood oxygenation level was tested using a commercial avoximeter (ITC, 1000E, Edison, N.J.). Blood with SaO₂=97.6% was placed in the PTFE tubing at room temperature for 230 min; after removal, SaO₂ level showed a 1.7% decrease to 95.9%.

Phase measurements provided by DWP-OCT are associated with optical pathlength (op) changes in response to dual-wavelength (765 nm and 800 nm) excitation of a blood sample. Optical pathlength changes (op=φλ/(4π)) in the sample are induced by light absorption of oxy- and deoxy-hemoglobin in blood, where φ is measured phase change at a specific depth and λ=1328 nm is central wavelength of the DWP-OCT system. Measurement of op at two laser excitation wavelengths is used to compute SaO₂ levels in blood using an analytical model described below.

Sample Preparation.

Arterial porcine blood was collected just before animal sacrifice. The blood was placed in a sealed container to prevent variation in SaO₂ levels and stored at 4° C. To prevent clotting, 50 units of heparin per 50 ml of blood were added immediately after collection. To prepare samples with variable SaO₂ levels for DWP-OCT measurement blood was divided into two equal parts and added to one portion 5 mg of Sodium Dithionite per 1 ml of blood to provide a sample with SaO₂=0%. The other part was bubbled with pure oxygen for fifteen minutes to saturate SaO₂ level. Next, the saturated oxygenated blood was mixed with the 0% SaO₂ blood in different proportions to achieve intermediate SaO₂ levels of 18.5%, 58.4%, 84.1% and 92.8%. Blood samples with desired SaO₂ levels were kept at room temperature in 2.5 ml sealed cuvettes for at least 20 min to avoid small drifts in oxygenation (Dalziel, 1957) during DWP-OCT measurements. The PTFE conduit with a 330 μm inner diameter and 480 μm outer diameter (SUBL-190, Braintree Scientific Inc.) was fastened to the top of a 1 mm thick glass slide using epoxy and filled with blood at a prepared SaO₂ level using a 1 ml syringe. Remaining blood in the syringe was utilized for immediate reference measurement of SaO₂ by the avoximeter. Manufacturer specified SaO₂ measurement precision of the avoximeter is 1%. After DWP-OCT measurement of each blood sample, the phantom vessel was flushed with distilled water and dried with forced air.

Laser Excitation.

A Mira™ 900 Ti:Al₂O₃ laser system (Coherent Inc.) was used in continuous wave mode to induce optical pathlength (op) changes in blood samples (FIG. 1A). First, the laser was tuned to oscillate at 765 nm as verified by an optical spectrometer (USB2000, Ocean Optics). Light emitted from the laser was coupled into a 0.22 NA 50 μm core-diameter multimode fiber using an optical collimator (f=11.23 mm). Light output from the fiber was collimated with a bi-convex lens (f=25.4 mm) and intensity modulated (fₒ=42 Hz) with an optical chopper. A small fraction (4%) of the light was utilized as an intensity reference and coupled into a Si photodetector (2032, New Focus) using a thin glass cover slip and lens (FIG. 1A). The intensity reference signal from the Si photodector was digitized with a 14-bit analog-to-digital converter (USB-6009, National Instruments) at 100 S/s and stored in computer memory for computation of SaO₂ levels.

The endface of the excitation laser output fiber was placed 1 mm below the glass slide underlying the blood sample giving a 900 μm beam-diameter on the vessel phantom. The relatively wide excitation beam diameter allowed easy co-registration of OCT and laser excitation beams. Light from the MIRA laser was blocked with a shutter when DWP-OCT data was not being recorded. To simultaneously irradiate the blood sample and record the intensity reference signal, the shutter was opened for 15-20 seconds and DWP-OCT data was recorded while excitation light (765 nm) impinged on the blood sample for 4-6 seconds. The measurement procedure using 765 nm excitation light was repeated three successive times for each blood sample. The MIRA laser was then tuned to 800 nm and the measurement procedure was repeated three successive times for the same blood sample with 765 nm excitation. Following laser excitation at 765 nm and 800 nm, blood in the phantom vessel was removed, the lumen cleaned and replaced with blood prepared at another SaO₂ level. The measurement procedure was repeated by exciting the blood sample at 765 nm and 800 nm and recording both DWP-OCT and laser excitation intensity reference data. Average laser excitation power at each blood sample was fixed at 23 mW (765 nm) and 51 mW (800 nm).

Phase Sensitive OCT System.

A phase sensitive (PhS) OCT system (FIG. 1) was used to measure nanometer scale changes in optical pathlength in the sample in response to laser excitation. The PhS-OCT system has been described in detail previously (Kuranov et al., 2010). Briefly, the PhS-OCT system uses a 20 kHz swept source laser with a central wavelength of 1328 nm and bandwidth of 100 nm (HSL-2000, Santec Corp.) and employs a common-path geometry. The system provides excellent phase stability (65 μm at a 280 μm depth) and low degradation of optical pathlength sensitivity with depth (0.16 nm mm). Acquisition and display of M-mode data uses a real-time uniform-frequency clock signal. An M-mode phase map and intensity A-scan of the phantom vessel filled with blood is shown in FIG. 2A and FIG. 2B, respectively.

Depth-Resolved Photothermal OCT Signal.

Depth-resolved Photothermal OCT signals corresponding to variation of optical pathlength (op) was measured at five depths in the blood sample (FIG. 2B): 1—the upper air-vessel interface (op=73 μm), 2—upper vessel-blood interface (op=187 μm), 3—lower blood-vessel interface (op=572 μm), 4—vessel-epoxy interface (op=676 μm), 5—epoxy-glass slide interface (op=749 μm). The intensity signal-to-noise ratio (SNR), magnitude of op change induced by excitation light near the isobestic point (800 nm) and SNR of the detected op at the five depths are summarized in Table 1.

TABLE 1
Typical signal and noise values at 5 depth positions (SaO₂ = 18%)
shown in FIG. 2. Here dB is computed as 20 · Log₁₀ (SNR).

| | Intensity | 765 nm | | 800 nm | |
|---|---|---|---|---|---|
| Depth | SNR (dB) | op amplitude (nm) | op SNR (dB) | op amplitude (nm) | op SNR (dB) |
| 1 | 70.4 ± 2.6 | 0.15 ± 0.0048 | 36.6 ± 1.1 | 0.24 ± 0.020 | 40.6 ± 1.5 |
| 2 | 50.3 ± 2.7 | 0.21 ± 0.11 | 12.5 ± 4.4 | 0.20 ± 0.059 | 12.9 ± 2.5 |
| 3 | 32.1 ± 4.0 | 1.21 ± 0.23 | 21.2 ± 0.96 | 1.70 ± 0.10 | 26.0 ± 4.4 |
| 4 | 48.3 ± 4.1 | 1.26 ± 0.065 | 36.5 ± 5.8 | 1.70 ± 0.071 | 43.1 ± 0.91 |
| 5 | 79.7 ± 3.78 | 1.29 ± 0.027 | 39.0 ± 7.8 | 1.74 ± 0.048 | 47.4 ± 1.2 |

At depth position 1 (upper air-vessel interface) op changes are induced by thermoelastic expansion of the blood and vessel walls. At depth position 2 (upper vessel-blood interface) op changes are induced by thermoelastic expansion of the blood and vessel wall and by thermorefractive effect (dn/dT) in the vessel wall. Since the sign, temporal profile (not shown) of op variation at depths 1 and 2 are equal within experimental error (Table 1) the inventors conclude that the phantom vessel walls do not absorb excitation light (765 nm and 800 nm) and op variation at depths 1 and 2 is primarily determined by thermoelastic expansion of the blood. Equivalent (within experimental error) op variation at depths 3 (lower blood-vessel interface), 4 (vessel-epoxy interface) and 5 (epoxy-glass slide interface) suggest light absorption in both the vessel wall and epoxy is negligible. Therefore, excitation-light induced op variation at depths 3, 4 and 5 are apparently due to thermorefractive and thermoelastic effects in blood. In as much as amplitude of op variation at depths 3, 4 and 5 are 8× larger than those at depths 1 and 2 (Table 1), the conclusion is that thermorefractive effect modifies op in blood by an order of magnitude greater than thermoelastic expansion.

Measured op changes at a given sample depth are the result of an accumulation of optical pathlength changes of probe light propagating through overlying layers (Paranjape et al., 2010). To measure SaO₂ levels in a blood sample, influence of optical pathlength changes in overlying layer(s) must be excluded and requires measurement of differential optical pathlength (Δop). In certain aspects, measurement of SaO₂ levels in the phantom vessel uses the Δop between the lower blood-vessel interface (depth 3) and the upper vessel-blood interface (depth 2) was computed. In a further aspect SaO₂ levels can be measured from a single depth. For reference, SaO₂ levels were also computed from Δop between depths 1 and 5, which provide higher SNR OCT signal intensities (Table 1). The magnitude of Δop at the upper air-vessel interface (depth 1) is equivalent (within experimental error) to that at the upper vessel-blood interface (depth 2) and magnitude of Δop at the lower blood-vessel interface (depth 3) is equivalent (within experimental error) to that at the epoxy-glass slide interface (depth 5) and vessel-epoxy interface (depth 4). DWP-OCT data was acquired during laser excitation (FIG. 3B). The slow drift component of optical pathlength is due to thermal transients in the phantom vessel and mounting components. After subtracting the linear trend, a time-segment (1s) of DWP-OCT data (FIG. 3C, between dashed lines in FIG. 3B) was Fourier transformed to compute the amplitude of differential optical pathlength at 42 Hz (Δop(fₒ=42 Hz), FIG. 3A black).

Blood Oxygenation (SaO₂) Levels.

Photothermal OCT is capable of measuring laser-induced variation in Δop on the nanometer scale in scattering objects such as human tissues (Paranjape et al., 2010; Adler et al., 2008; Skala et al., 2008; Zhou et al., 2010). In DWP-OCT, two laser excitation wavelengths are used to induce optical pathlength (op) changes in the sample. Difference in the absorption spectra between oxy- and deoxy-hemoglobin in two spectral regions (765 nm and 800 nm, see FIG. 4) may be utilized by DWP-OCT to determine blood oxygenation levels (SaO₂).

Since magnitude of op in response to clinically relevant excitation laser irradiance levels (mW and tens of mW) is small (0.2-2 nm), a linear relationship exists between differential optical pathlength (Δop) and fluence of excitation light absorbed by blood over a half-period (τ=½fₒ) of laser excitation:

Δop₁=kτI₁(1−e^{-μ}_{ₐ₁}^{l})

Δop₂=kτI₂(1−e^{-μ}_{ₐ₂}^{l}) (1),

where l—is vessel diameter, Δop₁—is differential optical pathlength between depths 2 and 3 (blood vessel) due to laser excitation at λ₁=765 nm, similarly, Δop₂—is differential optical pathlength between depths 2 and 3 (blood vessel) due to laser excitation at λ₂=800 nm, k—is a constant coefficient, I_{1(2)τ—fluence of} 765 nm (800 nm) excitation light on the phantom vessel, τ=½fₒ=0.012 second—is half period of modulation of excitation laser light, I₁ (I₂)—intensity amplitude of 765 nm (800 nm) excitation light incident on the phantom vessel, μₐ₁—is absorption coefficient of the blood sample at 765 nm and μₐ₂—is absorption coefficient of the blood sample at 800 nm. Neglecting absorption in blood by any constituent except for hemoglobin, gives algebraic expressions for the absorption coefficients of blood at 765 nm and 800 nm:

μₐ₁=α_{d1}c_{d}+αₒ₁cₒ

μₐ₂=α_{d2}c_{d}+αₒ₂cₒ (2),

where cₒ—is concentration of oxygenated hemoglobin (mM), c_{d}—is concentration of deoxygenated hemoglobin (mM), a_{o1,2}—tabulated molar extinction of oxygenated hemoglobin at λ₁=765 nm and λ₂=800 nm (cm⁻¹mM⁻¹, see FIG. 4), α_{d1,2}—tabulated molar extinction of deoxygenated hemoglobin at λ₁=765 nm and λ₂=800 nm (2) (cm⁻¹mM⁻¹, see FIG. 4). Equation (2) may be rewritten in terms of oxygen saturation (SaO₂=cₒ/THb) and total hemoglobin concentration (THb=cₒ+c_{d}):

μₐ₁=THb[SaO₂(αₒ₁−α_{d1})+α_{d1}]

μₐ₂=THb[SaO₂(αₒ₂−α_{d2})+α_{d2}] (3).

when absorption length of excitation light is much longer than the vessel diameter (μ_{a2,2}·lα·1), differential optical pathlength (Eq. 1) simplifies to,

Δop₁=kτd₁μₐ₁l

Δop₂=kτd₂μₐ₂l (4).

Blood oxygen saturation level (SaO₂) is obtained from the ratio Δop₁/Δop₂ and is written:

$$\begin{matrix}{SaO}_{2} = \frac{{α}_{d 1} - {χ}_{12} {α}_{d 2}}{{χ}_{12} ( {α}_{o 2} - {α}_{d 2} ) - ( {α}_{o 1} - {α}_{d 1} )} , \\ where & ( 5 ) \\ {χ}_{12} = \frac{Δ {op}_{1} {I}_{2}}{Δ {op}_{2} {I}_{1}} & ( 6 )\end{matrix}$$

By measuring differential optical pathlength (Δop) in blood at two wavelengths normalized by incident excitation light intensities, SaO₂ levels can be computed directly.

Average deviation of SaO₂ levels (Eq. 5) in the blood sample measured with DWP-OCT using Δop from depths 2 and 3 differ from the reference avoximeter values by less than 10.1% (FIG. 5). DWP-OCT measurements using Δop from depths 1 and 5, SaO₂ levels differ from reference values by less than 6.3%. Higher precision SaO₂ measurements using Δop between depths 5 and 1 as compare d to depths 3 and 2 is due to higher SNR of op at depth position 5 compared to 3 and depth position 1 compared to 2 (Table 1). The results demonstrate application of DWP-OCT to measure SaO₂ in a sample blood vessel.

The model proposed here assumes: (a) μₐₘₐₓl<<1, where μₐₘₐₓ is maximum absorption coefficient in the working range of excitation wavelengths and SaO₂ levels; (b) excitation wavelengths are sufficiently close so that optical throughput to the blood vessel at the two wavelengths is equal. Conditions (a) and (b) are satisfied here: (a) if SaO₂ levels are measured in a blood vessel with l=300 μm, maximal absorption coefficient μₐₘₐₓ=a_{d2}THb, where α_{d1}=1.435 cm⁻¹mM⁻¹ (Prahl, 1999), typical values in porcine blood (Craft and Moe, 1934) are THb=1.77 mM (11.4 g/dL) giving μₐₘₐₓl=0.76<<1 consistent with (a); (b) optical throughput of 765 nm and 800 nm light is equivalent because diffraction and scattering are nearly equal. Estimated temperature increase over a half period (on-time) of laser excitation is (Welch and van Gemert, 1995):

$$Δ T = \frac{{μ}_{α d 2} {l}_{02} τ}{{ρ}_{c}} = 0.14 , \qquad ( 7 )$$

where ΔT—is the maximal temperature increase due to absorption of excitation light by hemoglobin in the blood sample; μ_{ad2}=c_{d}α_{d2}=1.9 cm⁻² is absorption coefficient of deoxygenated blood (SaO₂=0%) at 800 nm, c_{d}=1.77 mM is typical concentration of deoxygenated hemoglobin in porcine blood (Craft and Moe, 1934), α_{d2}=0.816 cm⁻¹mM⁻¹ molar extinction of deoxygenated hemoglobin at 800 nm (Prahl, 1999); Iₐ₂τ—is a fluence of 800 nm excitation light on the blood sample during on period of the excitation cycle Iₐ₂=8 W/cm² intensity of 800 nm excitation light on the blood sample; x=4.8·10⁻² s is a half period of modulation of excitation laser light; ρ_{c}=cₚ·ρ=3.8 J·cm⁻²·K⁻² is volumetric heat capacity of blood, cₚ=3559 J·kg⁻¹K⁻¹ is specific heat capacity of blood (Petley et al., 2000), ρ=1060·10⁻⁶ kg·cm⁻² is density of the blood (Cutnell, 1997). Amplitude of op changes corresponding to ΔT are represented in Table 1. To estimate temperature increase ΔT₁ₛ over a 1 s time period, average optical pathlength op₁ₛ over one second was calculated:

$$Δ {T}_{1 s} = \frac{{op}_{1 s}}{op} Δ T = 0.66 \pm 0.31 . \qquad ( 8 )$$

Equations (5) and (6) indicate that measurement of SaO₂ requires that ratio of excitation light intensities (I₂/I₁) be monitored accurately. Since differential optical pathlength at the two laser excitation wavelengths (Δop₁ and Δop₂) was measured at fₒ=42 Hz, I₁ and I₂ were monitored by the Si photodetector at 42 Hz. Error of DWP-OCT measured SaO₂ levels (ΔSaO₂) depends on the SaO₂ level, relative errors in optical pathlength variation (δ[Δop_{1,2}]) and relative uncertainty in laser excitation intensities (δI) at the blood sample:

$$Δ {SaO}_{2} = {δχ}_{12} ( \frac{{α}_{d 1}}{{α}_{02} + {α}_{d 1} - {α}_{d 2} - {α}_{01}} - {SaO}_{2} ) \qquad ( 9 )$$

where α_{d2}=1.435 cm⁻¹mM⁻¹, αₒ₂=0.616 cm⁻¹ mm⁻¹, α_{d2}=0.762 cm⁻¹mM⁻¹, αₒ₂=0.816 cm⁻¹mM⁻1, δχ₂₂=δ[Δop₁]+δ[Δop₂]+2·δ1 is the relative error in parameter χ₁₂. Here relative error in optical pathlength δ[Δop₁]=(2.7±2.4) % and δ[Δop₂]=(0.4±0.2) % were derived from op measurements. Using measured SaO₂ values and their variation from reference values, relative uncertainty for 765 nm and 800 nm laser excitation intensities was assumed equal and is estimated at δ₁=(2.1±2.1) %.

Precision of DWP-OCT SaO₂ measurements can be improved by: (1) decreasing relative uncertainty of laser excitation intensity (δI) incident on the sample; (2) decreasing relative error in optical pathlength (δ[Δop]) by utilizing a higher modulation frequency of laser excitation thus detuning from low frequency phase drift artifacts. A modulation frequency of 42 Hz was selected in experiments reported here due to constraints of the mechanical chopper; (3) increase number of laser excitation wavelengths to more than two; or (4) increasing number of op measurements in the vessel wall at each laser excitation wavelength.

### II. Measurement of Total Hemoglobin

To measure total hemoglobin (THb) as following from Eq. 12 and 13 one needs to know thickness of the cuvette (measured with OCT) and intensity amplitude after the SaO₂ levels were calculated from ratio of measured op's at excitation wavelengths. Coefficient k can be calculated from dn/dT. The equation for the k can be derived from comparing Eq 13 and 16. Or alternatively THb can be calculated by monitoring intensity of the transmitted light through the cuvette.

$$\begin{matrix}Δ T = \frac{{μ}_{a 1 , 2} {I}_{01 , 2} τ}{{ρ}_{c}} & ( 10 ) \\ {μ}_{a 1 , 2} = THb [ {SaO}_{2} ( {α}_{o 1 , 2} - {α}_{d 1 , 2} ) + {α}_{d 1 , 2} ] & ( 11 ) \\ Δ T = \frac{{I}_{o 1 , 2} τ THb [ {SaO}_{2} ( {α}_{o 1 , 2} - {α}_{d 1 , 2} ) + {α}_{d 1 , 2} ]}{{ρ}_{c}} & ( 12 ) \\ Δ n = ( \frac{d n}{d T} ) Δ T & ( 13 ) \\ Δ op = 2 k Δ nl & ( 14 ) \\ THb = \frac{{λρ}_{c} Δ op}{4 π ( \frac{d n}{d T} ) {I}_{o 1 , 2} τ Thb [ {SaO}_{2} ( {α}_{o 1 , 2} - {α}_{d 1 , 2} ) + {α}_{d 1 , 2} ] l} & ( 15 )\end{matrix}$$

In the case one cannot calibrate sufficiently one of the parameters one would proceed with

$$THb = \frac{{γλρ}_{c} Δ op}{4 π ( \frac{d n}{d T} ) {I}_{o 1 , 2} τ Thb [ {SaO}_{2} ( {α}_{o 1 , 2} - {α}_{d 1 , 2} ) + {α}_{d 1 , 2} ] l} \qquad ( 16 )$$

And one time calibrated γ using one of the precise lab devices measuring THb.

One of the factors that influenced the precision of the DWP-OCT for measurements of the hemoglobin species is the signal to noise ratio of the intensity OCT signal. The intensity OCT signal can be measured from one of the interfaces of cuvette numbered from 1 to 4 (1—external surface of proximal cuvette wall; 2—internal surface of proximal cuvette wall; 3—internal surface of distal cuvette wall; and 4—external surface or distal cuvette wall; see FIG. 6). The sample will be positioned between the cuvette walls. The natural choice for the measurements of differential optical pathlength (op) variation to be used in calculations of concentrations of the hemoglobin species is op23 between internal surfaces of the cuvette, position 2 and 3. But, because of the close matching of refractive indexes of the blood and polymer or glass, the SNR of the intensity OCT signal can be reduced from those boundaries. Here there are several alternatives to improve intensity OCT SNR and therefore increase the precision of the measurements:

1. Make surface 2 partially reflecting using metal or polymer thin film coating (range of reflection 5-50%)+make surface 3 100% reflecting using metal or polymer thin film coating.

2. The bulk material of the cuvette can be selected so as to not absorb any of the excitation wavelength and in this case one can measure op14 for calculation where air/cuvette interface provide higher reflection due to higher refractive index mismatch.

3. Any of the variations of the following op variations can be used: op13, op23, op14, op24.

4. When using op13 coat surface 1 (5-50% reflection), surface 3 100% reflection; when using 23 surface 2 5-50% reflection, surface 3 100% reflection; when using op14 surface 1 5-50% reflection, surface 4 100% reflection; when using op24 surface 2 5-50% reflection, surface 4 100% reflection.

For simultaneous measurement of the five hemoglobin species concentrations five simultaneous excitation wavelength are needed with distinct modulation frequencies:

$$\{ \begin{matrix}{μ}_{a 1} = {c}_{d} {α}_{d 1} + {c}_{o} {α}_{o 1} + {c}_{c} {α}_{c 1} + {c}_{m} {α}_{m 1} + {c}_{s} {α}_{s 1} \\ … = … \\ {μ}_{a 5} = {c}_{d} {α}_{d 5} + {c}_{o} {α}_{o 5} + {c}_{c} {α}_{c 5} + {c}_{m} {α}_{m 5} + {c}_{s} {α}_{s 5}\end{matrix} \qquad ( 17 )$$

where c_{d}—is concentration of deoxyhemoglobin, cₒ—is concentration of oxyhemoglobin, c_{c}—is concentration of carboxyhemoglobin, cₘ—is concentration of methemoglobin and cₛ—is concentration of sulfhemoglobin, a_{di}—tabulated molar extinction of deoxyhemoglobin at i-th excitation wavelength where i=1, 2 . . . 5, aₒᵢ—tabulated molar extinction of oxyhemoglobin, α_{ci}—tabulated molar extinction of carboxyhemoglobin, αₘᵢ—tabulated molar extinction of methemoglobin, α_{ci}—tabulated molar extinction of sulfhemoglobin.

The equation 17 can rewritten in more compact form:

μₐᵢ=c_{d}α_{di}+cₒαₒᵢ+c_{c}α_{ci}+cₘαₘᵢ+cₛαₛᵢ (18).

DWP OCT measuring excitation induced optical pathlengths at i-th wavelength (Δopᵢ) changes:

$$Δ {op}_{i} = \frac{4 π}{λ} Δ {n}_{i} l \qquad ( 19 )$$

where λ—probing light wavelength, Δnᵢ—is the refractive index variation, l—thickness of the cuvette (the thickness of the cuvette can be measured very precisely with the Intensity OCT). Refractive index change depends on the absorption of the blood:

$$Δ {n}_{i} = {( \frac{d n}{d T} )}_{i} Δ {T}_{i} = {( \frac{d n}{d T} )}_{i} \frac{{μ}_{ai} {I}_{oi} τ}{{ρ}_{c}} . \qquad ( 20 )$$

where I₀ᵢτ—is a fluence of i-th excitation light wavelength on the blood sample during half period of the excitation cycle, I₀₂ is intensity of excitation light on the blood sample; τ is a half period of modulation of excitation laser light; ρ_{c}=cₚ·ρ=3.8 J·cm⁻³·K⁻¹ is volumetric heat capacity of blood, cₚ is specific heat capacity of blood, ρ=1060·10⁻⁶ kg⁻¹·cm⁻³ is density of the blood. The measured Δopᵢ variations therefore depend on absorption coefficients of blood:

$$Δ {op}_{i} = \frac{4 π}{λ} {( \frac{d n}{d T} )}_{i} \frac{{μ}_{ai} {I}_{oi} τ}{{ρ}_{c}} l \qquad ( 21 )$$

The equation 21 in extended form:

$$\{ \begin{matrix}\frac{{λρ}_{c} γ}{4 π l τ} Δ {op}_{1} = {{I}_{o 1} ( \frac{d n}{d T} )}_{1} {α}_{d 1} {c}_{d} + {{I}_{o 1} ( \frac{d n}{d T} )}_{1} {α}_{o 1} {c}_{o} + {{I}_{o 1} ( \frac{d n}{d T} )}_{1} {α}_{c 1} {c}_{c} + {{I}_{o 1} ( \frac{d n}{d T} )}_{1} {α}_{m 1} {c}_{m} + {{I}_{o 1} ( \frac{d n}{d T} )}_{1} {α}_{s 1} {c}_{s} \\ \frac{{λρ}_{c} γ}{4 π l τ} Δ {op}_{2} = {{I}_{o 2} ( \frac{d n}{d T} )}_{2} {α}_{d 2} {c}_{d} + {{I}_{o 2} ( \frac{d n}{d T} )}_{2} {α}_{o 2} {c}_{o} + {{I}_{o 2} ( \frac{d n}{d T} )}_{2} {α}_{c 2} {c}_{c} + {{I}_{o 2} ( \frac{d n}{d T} )}_{2} {α}_{m 2} {c}_{m} + {{I}_{o 2} ( \frac{d n}{d T} )}_{2} {α}_{s 2} {c}_{s} \\ \frac{{λρ}_{c} γ}{4 π l τ} Δ {op}_{3} = {{I}_{o 3} ( \frac{d n}{d T} )}_{3} {α}_{d 3} {c}_{d} + {{I}_{o 3} ( \frac{d n}{d T} )}_{3} {α}_{o 3} {c}_{o} + {{I}_{o 3} ( \frac{d n}{d T} )}_{3} {α}_{c 3} {c}_{c} + {{I}_{o 3} ( \frac{d n}{d T} )}_{3} {α}_{m 3} {c}_{m} + {{I}_{o 3} ( \frac{d n}{d T} )}_{3} {α}_{s 3} {c}_{s} \\ \frac{{λρ}_{c} γ}{4 π l τ} Δ {op}_{4} = {{I}_{o 4} ( \frac{d n}{d T} )}_{4} {α}_{d 4} {c}_{d} + {{I}_{o 4} ( \frac{d n}{d T} )}_{4} {α}_{o 4} {c}_{o} + {{I}_{o 4} ( \frac{d n}{d T} )}_{4} {α}_{c 4} {c}_{c} + {{I}_{o 4} ( \frac{d n}{d T} )}_{4} {α}_{m 4} {c}_{m} + {{I}_{o 4} ( \frac{d n}{d T} )}_{4} {α}_{s 4} {c}_{s} \\ \frac{{λρ}_{c} γ}{4 π l τ} Δ {op}_{5} = {{I}_{o 5} ( \frac{d n}{d T} )}_{5} {α}_{d 5} {c}_{d} + {{I}_{o 5} ( \frac{d n}{d T} )}_{5} {α}_{o 5} {c}_{o} + {{I}_{o 5} ( \frac{d n}{d T} )}_{5} {α}_{c 5} {c}_{c} + {{I}_{o 5} ( \frac{d n}{d T} )}_{5} {α}_{m 5} {c}_{m} + {{I}_{o 5} ( \frac{d n}{d T} )}_{5} {α}_{s 5} {c}_{s}\end{matrix} \qquad ( 22 )$$

Here correction coefficient γ may be used or not. The γ should be experimentally determined once for current geometry.

Using, for example, Cramer's rule for solving system of linear equations one's can calculate the concentrations of the hemoglobin species:

$$A = deT [ \begin{matrix}{{I}_{d 1} ( \frac{d n}{d T} )}_{1} {α}_{d 1} & {{I}_{o 1} ( \frac{d n}{d T} )}_{1} {α}_{o 1} & {{I}_{o 1} ( \frac{d n}{d T} )}_{1} {α}_{c 1} & {{I}_{o 1} ( \frac{d n}{d T} )}_{1} {α}_{m 1} & {{I}_{o 1} ( \frac{d n}{d T} )}_{1} {α}_{s 1} \\ {{I}_{d 2} ( \frac{d n}{d T} )}_{2} {α}_{d 2} & {{I}_{o 2} ( \frac{d n}{d T} )}_{2} {α}_{o 2} & {{I}_{o 2} ( \frac{d n}{d T} )}_{2} {α}_{c 2} & {{I}_{o 2} ( \frac{d n}{d T} )}_{2} {α}_{m 2} & {{I}_{o 2} ( \frac{d n}{d T} )}_{2} {α}_{s 2} \\ {{I}_{d 3} ( \frac{d n}{d T} )}_{3} {α}_{d 3} & {{I}_{o 3} ( \frac{d n}{d T} )}_{3} {α}_{o 3} & {{I}_{o 3} ( \frac{d n}{d T} )}_{3} {α}_{c 3} & {{I}_{o 3} ( \frac{d n}{d T} )}_{3} {α}_{m 3} & {{I}_{o 3} ( \frac{d n}{d T} )}_{3} {α}_{s 3} \\ {{I}_{d 4} ( \frac{d n}{d T} )}_{4} {α}_{d 4} & {{I}_{o 4} ( \frac{d n}{d T} )}_{4} {α}_{o 4} & {{I}_{o 4} ( \frac{d n}{d T} )}_{4} {α}_{c 4} & {{I}_{o 4} ( \frac{d n}{d T} )}_{4} {α}_{m 4} & {{I}_{o 4} ( \frac{d n}{d T} )}_{4} {α}_{s 4} \\ {{I}_{d 5} ( \frac{d n}{d T} )}_{5} {α}_{d 5} & {{I}_{o 5} ( \frac{d n}{d T} )}_{5} {α}_{o 5} & {{I}_{o 5} ( \frac{d n}{d T} )}_{5} {α}_{c 5} & {{I}_{o 5} ( \frac{d n}{d T} )}_{5} {α}_{m 5} & {{I}_{o 5} ( \frac{d n}{d T} )}_{5} {α}_{s 5}\end{matrix} ] \\ A 1 = deT [ \begin{matrix}\frac{{λρ}_{c} γ}{4 π l τ} Δ {op}_{1} & {{I}_{o 1} ( \frac{d n}{d T} )}_{1} {α}_{o 1} & {{I}_{o 1} ( \frac{d n}{d T} )}_{1} {α}_{c 1} & {{I}_{o 1} ( \frac{d n}{d T} )}_{1} {α}_{m 1} & {{I}_{o 1} ( \frac{d n}{d T} )}_{1} {α}_{s 1} \\ \frac{{λρ}_{c} γ}{4 π l τ} Δ {op}_{2} & {{I}_{o 2} ( \frac{d n}{d T} )}_{2} {α}_{o 2} & {{I}_{o 2} ( \frac{d n}{d T} )}_{2} {α}_{c 2} & {{I}_{o 2} ( \frac{d n}{d T} )}_{2} {α}_{m 2} & {{I}_{o 2} ( \frac{d n}{d T} )}_{2} {α}_{s 2} \\ \frac{{λρ}_{c} γ}{4 π l τ} Δ {op}_{3} & {{I}_{o 3} ( \frac{d n}{d T} )}_{3} {α}_{o 3} & {{I}_{o 3} ( \frac{d n}{d T} )}_{3} {α}_{c 3} & {{I}_{o 3} ( \frac{d n}{d T} )}_{3} {α}_{m 3} & {{I}_{o 3} ( \frac{d n}{d T} )}_{3} {α}_{s 3} \\ \frac{{λρ}_{c} γ}{4 π l τ} Δ {op}_{4} & {{I}_{o 4} ( \frac{d n}{d T} )}_{4} {α}_{o 4} & {{I}_{o 4} ( \frac{d n}{d T} )}_{4} {α}_{c 4} & {{I}_{o 4} ( \frac{d n}{d T} )}_{4} {α}_{m 4} & {{I}_{o 4} ( \frac{d n}{d T} )}_{4} {α}_{s 4} \\ \frac{{λρ}_{c} γ}{4 π l τ} Δ {op}_{5} & {{I}_{o 5} ( \frac{d n}{d T} )}_{5} {α}_{o 5} & {{I}_{o 5} ( \frac{d n}{d T} )}_{5} {α}_{c 5} & {{I}_{o 5} ( \frac{d n}{d T} )}_{5} {α}_{m 5} & {{I}_{o 5} ( \frac{d n}{d T} )}_{5} {α}_{s 5}\end{matrix} ] \\ A 2 = deT [ \begin{matrix}{{I}_{d 1} ( \frac{d n}{d T} )}_{1} {α}_{d 1} & \frac{{λρ}_{c} γ}{4 π l τ} Δ {op}_{1} & {{I}_{o 1} ( \frac{d n}{d T} )}_{1} {α}_{c 1} & {{I}_{o 1} ( \frac{d n}{d T} )}_{1} {α}_{m 1} & {{I}_{o 1} ( \frac{d n}{d T} )}_{1} {α}_{s 1} \\ {{I}_{d 2} ( \frac{d n}{d T} )}_{2} {α}_{d 2} & \frac{{λρ}_{c} γ}{4 π l τ} Δ {op}_{2} & {{I}_{o 2} ( \frac{d n}{d T} )}_{2} {α}_{c 2} & {{I}_{o 2} ( \frac{d n}{d T} )}_{2} {α}_{m 2} & {{I}_{o 2} ( \frac{d n}{d T} )}_{2} {α}_{s 2} \\ {{I}_{d 3} ( \frac{d n}{d T} )}_{3} {α}_{d 3} & \frac{{λρ}_{c} γ}{4 π l τ} Δ {op}_{3} & {{I}_{o 3} ( \frac{d n}{d T} )}_{3} {α}_{c 3} & {{I}_{o 3} ( \frac{d n}{d T} )}_{3} {α}_{m 3} & {{I}_{o 3} ( \frac{d n}{d T} )}_{3} {α}_{s 3} \\ {{I}_{d 4} ( \frac{d n}{d T} )}_{4} {α}_{d 4} & \frac{{λρ}_{c} γ}{4 π l τ} Δ {op}_{4} & {{I}_{o 4} ( \frac{d n}{d T} )}_{4} {α}_{c 4} & {{I}_{o 4} ( \frac{d n}{d T} )}_{4} {α}_{m 4} & {{I}_{o 4} ( \frac{d n}{d T} )}_{4} {α}_{s 4} \\ {{I}_{d 5} ( \frac{d n}{d T} )}_{5} {α}_{d 5} & \frac{{λρ}_{c} γ}{4 π l τ} Δ {op}_{5} & {{I}_{o 5} ( \frac{d n}{d T} )}_{5} {α}_{c 5} & {{I}_{o 5} ( \frac{d n}{d T} )}_{5} {α}_{m 5} & {{I}_{o 5} ( \frac{d n}{d T} )}_{5} {α}_{s 5}\end{matrix} ] \\ A 3 = deT [ \begin{matrix}… & … & \frac{{λρ}_{c} γ}{4 π l τ} Δ {op}_{1} & … & … \\ … & … & \frac{{λρ}_{c} γ}{4 π l τ} Δ {op}_{2} & … & … \\ … & … & \frac{{λρ}_{c} γ}{4 π l τ} Δ {op}_{3} & … & … \\ … & … & \frac{{λρ}_{c} γ}{4 π l τ} Δ {op}_{4} & … & … \\ … & … & \frac{{λρ}_{c} γ}{4 π l τ} Δ {op}_{5} & … & …\end{matrix} ] , A 4 = deT [ \begin{matrix}… & … & … & \frac{{λρ}_{c} γ}{4 π l τ} Δ {op}_{1} & … \\ … & … & … & \frac{{λρ}_{c} γ}{4 π l τ} Δ {op}_{2} & … \\ … & … & … & \frac{{λρ}_{c} γ}{4 π l τ} Δ {op}_{3} & … \\ … & … & … & \frac{{λρ}_{c} γ}{4 π l τ} Δ {op}_{4} & … \\ … & … & … & \frac{{λρ}_{c} γ}{4 π l τ} Δ {op}_{5} & …\end{matrix} ] , \\ A 5 = deT [ \begin{matrix}… & … & … & … & \frac{{λρ}_{c} γ}{4 π l τ} Δ {op}_{1} \\ … & … & … & … & \frac{{λρ}_{c} γ}{4 π l τ} Δ {op}_{2} \\ … & … & … & … & \frac{{λρ}_{c} γ}{4 π l τ} Δ {op}_{3} \\ … & … & … & … & \frac{{λρ}_{c} γ}{4 π l τ} Δ {op}_{4} \\ … & … & … & … & \frac{{λρ}_{c} γ}{4 π l τ} Δ {op}_{5}\end{matrix} ] \\ {c}_{d} = \frac{A 1}{A} , {c}_{o} = \frac{A 2}{A} , {c}_{c} = \frac{A 3}{A} , {c}_{m} = \frac{A 4}{A} , {c}_{s} = \frac{A 5}{A} . \qquad ( 23 )$$

The other approaches for solving system of linear equations can be used. Here deT is a determinant of the matrix.

Finally the total hemoglobin (THb) and relative amount of species can be calculated as follows:

$$THb = {c}_{d} + {c}_{o} + {c}_{c} + {c}_{m} + {c}_{s} \\ {SaO}_{2} = \frac{{c}_{o}}{THB} \\ SaD = \frac{{c}_{d}}{THB} \\ SaC = \frac{{c}_{c}}{THB} \\ SaM = \frac{{c}_{m}}{THB} \\ SaS = \frac{{c}_{s}}{THB} . \qquad ( 24 )$$

where SaO₂—relative amount of the oxyhemoglobin (oxygenation level of the blood), SaD—relative amount of the deoxyhemoglobin, SaC—relative amount of the carboxyhemoglobin, SaM—relative amount of the methemoglobin, SaS—relative amount of the sulfhemoglobin.

### III. In Vivo Depth-Resolved Oxygen Saturation by Dual-Wavelength Photothermal (DWP) OCT

Calculation of SaO₂ Levels.

DWP-OCT phase data recorded over a 10 s time period (FIG. 9) corresponding to light reflected from the posterior side of a 30 μm diameter target arteriole was used to calculate SaO₂ levels. Diameter of the arteriole was measured from a recorded digital image (FIG. 7B). Depth location of the back reflected light from the target arteriole was determined from Doppler (Chen et al., 1997; Leitgeb et al., 2003) and speckle contrast (Barton and Stromski, 2005; Mariampillai et al., 2008) images (FIG. 9). Speckle contrast M-mode images were acquired using the Mariampillai et al. (2008) algorithm. Each A-scan in the speckle contrast Mmode image was calculated as a standard deviation between four adjacent OCT-signal (intensity) A-scans. The Doppler M-mode image was acquired using the Leitgeb et al. (2003) algorithm. To calculate each A-scan in the Doppler M-mode image the inventors start from 512 phase A-scans and calculate the average difference between five consecutive phase A-scans.

Modulations observed in recorded phase data (FIG. 9) were those of murine heart and breathing rates. To acquire op variations associated with absorption of photothermal excitation light by blood the 10 s segment of phase data was divided into 21 segments each 5 long and separated by 0.25 s: the first 5 s segment starts at 0 s while 21st segment starts at 5 within the 10 s segment. Fast Fourier transformation of the 5 s phase data segments (FIG. 10) revealed op variations with amplitude ranging 1-2 nm at f₁=400 Hz and f₂=380 Hz due to absorption of photothermal excitation light by blood in the target arteriole. The normalized op₁/op₂ ratio (χ₁₂=(op₁/Φ₁)/(op₂/Φ₂)=I₂f₁op₁/(I₁f₂op₂)) was used to calculate SaO₂ levels (Eq. (25)). The ratio I₂/I₁ was determined from the average power of photothermal excitation light at 770 nm and 800 nm. Average radiant power at the DWP-OCT probe was measured with a calibrated power meter (1936-C, Newport, Irvine, Calif.) to give 8.7 mW at 770 nm and 10.7 mW at 800 nm.

Phase data in each 5 s segment was processed when OCT intensity signal variation was less than 15 dB (FIG. 9). SaO₂ level determined from DWP-OCT was calculated as a mean of 21 values from each of the 5 s time segments.

Optimizing Intensity Modulation Frequencies.

Selection of optimal intensity modulation frequencies was done for robust SaO₂ measurement. Optimal modulation frequency (fₘₒₚₜ=400 Hz) was experimentally determined based on the maximal ratio of op_{SNR}/op_{SD} (FIG. 11), where op_{SNR}=op₂/opₙₒᵢₛₑ and opₙₒᵢₛₑ was standard deviation (n=20) of op background amplitude, op_{SD} is standard deviation of op_{SNR} over five consecutive measurements. Here, op₂ was used to determine optimal intensity modulation frequency since light absorption at 800 nm had a much weaker dependence on SaO₂ levels compared to op₁ (770 nm). Signal-to-noise ratio of optical pathlength variation (op_{SNR}) at fₘₒₚₜ=400 Hz was nearly equal to maximal op_{SNR} (fₘ=600 Hz) only with a substantially smaller standard deviation (FIG. 11).

Gas Challenge Verification of DWP-OCT in Murine Brain Arteriole.

Calculated SaO₂ levels from DWP-OCT phase data for each of the twenty one 5-s segments measured in a target arteriole in the murine brain are shown in FIG. 12. Systemic SaO₂ levels measured with a pulse-oximeter are indicated in each plot. Calculated SaO₂ levels from DWPOCT phase data revealed oscillatory temporal dynamics during every time segments at fixed reference SaO₂ levels. Average DWP-OCT SaO₂ values are plotted versus systemic pulseoximeter SaO₂ values (FIG. 13).

The DWP-OCT SaO₂ values were linearly correlated (R₂=0.98, n=5) and slightly and systematically lower than arterial blood-gas SaO₂ except for the blood-gas SaO₂=99% when the animal inhaled pure oxygen. In calculations of R₂ blood-gas SaO₂ value of 99% was excluded. Systemic arterial SaO₂ values are known to be higher than brain arteriole's SaO₂ values due to gas exchange between arteriole blood and surrounding tissue (Vovenko, 1999). When the animal breathed pure oxygen the DWP-OCT (100.4%) as well as blood-gas (99%) SaO₂ values showed that the systemic and arteriole's blood hemoglobin were totally oxygenated within experimental error. The inventors contemplate that during pure oxygen inhalation, blood plasma contains excess dissolved oxygen sufficient to both diffuse to surrounding tissue and saturate (100%) arteriole hemoglobin. The averaged difference between DWP-OCT and systemic pulse-oximeter SaO₂ values was 10.1% with the pure oxygen breathing measurement included and 11.1% without. The standard error of measurements of 2.1% is estimated from 4.3% residual mean square of a linear fit of the 5 lowest SaO₂ values presented in FIG. 13.

DWP-OCT System for In-Vivo Measurements.

The experimental setup for our DWP-OCT system (FIG. 7) to measure SaO₂ levels contains two major components: (a) photothermal excitation lasers at 800 nm and 770 nm to induce nanometer-scale optical pathlength (op) changes in murine tissue; and (b) a Phase Sensitive (PhS) OCT system (Kuranov et al., 2010) to measure SaO₂-dependent op changes induced by photothermal excitation laser light.

Depth-resolved phase measurements in tissue provided by DWP-OCT are associated with op changes in response to dual-wavelength (770 nm and 800 nm) photothermal excitation of blood in a target microvessel. Optical pathlength changes (op=φλ/(4π)) in the murine brain are induced by light absorption of oxy- and deoxy-hemoglobin in a microvessel, where φ is measured phase change at a specific depth and λ=1328 nm is central wavelength of the tunable laser source. Simultaneous measurement of op in response to 770 nm and 800 nm laser photothermal excitation wavelengths is used to compute SaO₂ levels in blood using an analytical model for χ₁₂ described in phantom studies (Kuranov et al., 2011).

Phase Sensitive (PhS) OCT System.

A common-path Phase Sensitive (PhS) OCT system was used to measure nanometer scale changes in optical pathlength (op) in the murine brain in response to laser photothermal excitation. The PhS-OCT system has been described in Kuranov et al., 2010. Briefly, the PhS-OCT system uses a 20 kHz polygon mirror tunable laser (HSL-2000, Santec USA Corp., Hackensack, N.J.) with a central wavelength of 1328 nm, bandwidth of 100 nm and a measured depth resolution of 16 μm in air. The system provides excellent phase stability in transparent (65 pm at a 280 μm depth) and scattering media (less than 1 nm up to 864 μm depth) and low degradation of optical pathlength sensitivity with increasing depth (0.16 nm mm in transparent and 2.8 nm mm in scattering media).

The PhS-OCT system used SMF-28 fiber (Corning Inc., Corning, N.Y.) and contains four subsystems: (1) common-path sample and (2) reference interferometers; (3) a gas-cell based spectral trigger; and (4) real-time Mach-Zehnder external clock interferometer. In the signal interferometer interference fringes formed between light reflected from the end-face of a right-angle cleaved single-mode fiber and the murine brain tissue. A Hydrogen fluoride (HF) fiber-coupled gas-cell (HF-50, Wavelength Reference, Corvallis, Oreg.) provided a temperature/pressure-independent trigger wavelength (λₛₜ=1272.97030 nm) for each A-scan. A repeatable and fixed trigger wavelength guaranteed a fixed phase φ=opλₛₜ/(4π) for each A-scan for a fixed optical pathlength (op) in the sample up to an uncertainty of one external clock period. A reference interferometer was implemented to remove the one clock period uncertainty.

A uniform-frequency external clock was implemented to compensate for dispersion effects associated with the nonlinear sweep rate of the tunable laser that would have resulted in degradation of the point spread function and reduced SNR with increasing scan depth (Choma et al, 2005; Yun et al., 2003). Since the tunable laser used in this study has a 65% duty cycle, uninterrupted real-time acquisition and display of intensity (FIG. 7C) and speckle contrast (FIG. 8A) M-mode data (39 framess of 512×400 (intensity) and 128×400 (speckle contrast) pixels) was realized by timely substitution (over 35% of duty cycle off-time) of the uniform-frequency optical clock with single-frequency electrical pseudo-clock (Kuranov et al., 2010).

Laser Photothermal Excitation and Photothermal OCT Signal.

Photothermal excitation beams at 770 nm and 800 nm were combined with the 1328 nm DWP-OCT probe beam in a common optical fiber (Corning SMF-28) using a 8001310 nm wavelength-division multiplexer (WDM-1300-800-SP, Thorlabs, Newton, N.J.). SMF-28 optical fiber is single-mode at 1328 nm (OCT) and supports propagation of a few modes at photothermal excitation wavelengths (770 nm and 800 nm). Photothermal excitation light emitted from two 100 mW single-mode fiber pigtailed laser diodes (QPhotonics, LLC, Ann Arbor, Mich.: QFLD-780-100S for 770 nm and QFLD-808-100S for 800 nm) were combined in a fiber coupler (Optowaves Inc., San Jose, Calif.). To maintain a stable emission wavelength at 770 nm and 800 nm, temperature of the diode lasers was fixed at approximately 278 K with 0.01 K precision using temperature controllers (TED200C, Thorlabs, Newton, N.J.). Emission wavelengths of the diode lasers were verified by an optical spectrometer (USB2000, Ocean Optics, Dunedin, Fla.).

To study in vivo microvasculature, DWP-OCT photothermal excitation and probe beams must be incident on tissue from a common side. The common path photothermal excitationprobe geometry insured single-sided and co-registration of photothermal excitation and probe beams on a target arteriole in the murine brain and increased DWP-OCT signal amplitude compared to phantom experiments (Kuranov et al., 2011). Probe and photothermal excitation beams were incident on the target arteriole directly from the endface of the SMF-28 fiber without any intervening optics. The DWP-OCT probe fiber was cleaved at a right angle to provide a 4% backreflection that was used as the reference signal for the commonpath sample interferometer (Kuranov et al., 2010).

Computation of SaO₂ levels in the microvessel are based on the assumption of a linear relationship between optical pathlength variations (op) and fluence of photothermal excitation light absorbed by microvessel blood over a period (τ₁₍₂₎) of laser excitation (Welch and van Gemert, 1995):

op₁=kτ₁I₁(1−e^{-μ}^{ₐ₁}^{l}),

op₂=kτ₂I₂(1−e^{-μ}^{ₐ₂}^{l}), (25)

where τ₁₍₂₎ is the period of photothermal excitation (subscript 1 denote laser excitation at λ₁=770 nm and subscript 2 at λ₂=800 nm); Φ1(2)=I_{1(2)τ1(2)}—fluence and I₁₍₂₎ intensity amplitude of excitation light on the microvessel, l=20 μm is the microvessel's lumen diameter (FIG. _), op₁₍₂₎ is amplitude of optical pathlength variation, k—a constant depending on mechanical properties of the tissue, μₐ₁₍₂₎—absorption coefficient of the blood: μₐ₁₍₂₎=α_{d1(2)}c_{d}+αₒ₁₍₂₎cₒ, where cₒ and c_{d} are concentrations of oxygenated and deoxygenated hemoglobin (mM), αₒ₁₍₂₎ and α_{d1(2)} are tabulated molar extinction of oxygenated and deoxygenated hemoglobin (cm⁻¹ mM⁻¹): αₒ₁=0.650 cm⁻¹ mM⁻¹, αo2=0.816 cm⁻¹ mM⁻¹, α_{d1}=1.312 cm⁻¹ mM⁻¹, α_{d2}=0.762 cm⁻¹ mM⁻¹ (Prahl 1999).

The parameter μₐ₁₍₂₎1 achieves its maximal value of α_{d1}c_{d}_{₁}=4.6×10⁻³ (c_{d}=1.77 mM is typical concentration of hemoglobin in mammalian blood) when 770 nm photothermal excitation laser induce op variations in a totally deoxygenated microvessel (SaO₂=0%). Hemoglobin concentration in mM was calculated from the conversion value of 11.4 gdL (Craft and Moe, 1934) using hemoglobin molecular weight MW=64500 g/mole. Since μₐ₁₍₂₎1<<1 for microvessels we obtained SaO₂=cₒ/(cₒ+c_{d}) from the op₁/op₂ ratio as Kuranov et al., 2011:

$${SaO}_{2} = \frac{{α}_{d 1} - {χ}_{12} {α}_{d 2}}{{χ}_{12} ( {α}_{o 2} - {α}_{d 2} ) - ( {α}_{o 1} - {α}_{d 1} )} . \qquad ( 26 )$$

where χ₁₂=(op₁/Φ₁)/(op₂/Φ₂) is the normalized ratio of op variation. The Eq. (26) shows that SaO₂ levels can be computed directly by measuring op at two wavelengths normalized by incident excitation light fluences.

To remove ambiguity in calculation of SaO₂ levels associated with temporal variation in the blood layer thickness (microvessel lumen, 1) and total hemoglobin concentration (THb=cₒ+c_{d}) in the target arteriole, we implemented simultaneous dual-wavelength (770 nm and 800 nm) photothermal excitation (FIG. 7). As can be seen from Eq. (27) in the case when 1 and THb are functions of time (l=l(t) and THb=THb(t)) the parameter χ₁₂ used for calculation of SaO₂ levels in Eq. (26) can vary unpredictably over time (Δt=t2−t₁):

op₁/Φ₁=k[SaO₂(αₒ₁−α_{d1})+α_{d1}]THb(t)l(t),

op₂/Φ₂=k[SaO₂(αₒ₂−α_{d2})+α_{d2}]THb(t)l(t), (27)

where t₁₍₂₎ is time of measurement of op₁₍₂₎. Equation 27 is Eq. 25 written in terms of SaO₂ and Thb after simplification.

Temporal variation of in vivo blood layer thickness [l=l(t)] is due to arteriole pulsation associated with cardiac and respiratory motion. Respiratory motion may lead to temporal variation in 1 by lateral movement of the target arteriole with respect to DWP-OCT probe and photothermal excitation beams. Total hemoglobin concentration (THb) can vary with time in response to an intravascular injection due to transient blood dilution. Separation between op₁ (770 nm) and op₂ (800 nm) was achieved using frequency encoding by intensity modulation of the two photothermal excitation beams (770 nm at f₁=400 Hz and 800 nm at f₂=380 Hz). Modulation of photothermal excitation beams was achieved by modulating laser diode driver's current (505B, Newport Corp., Irvine, Calif.) with a pure sinusoidal voltage waveform using two distinct arbitrary waveform generators (33250A, Agilent Technologies Inc., Santa Clara, Calif.).

Animal Preparation.

Experiments were performed in accordance with the University of Texas at Austin IACUC protocols. Adult male mice (30 g, strain: CD-1, Charles River Laboratories International, Inc., Wilmington, Mass.) were anesthetized with 2% isoflurane in a 70/30 N₂O/O₂ gas mixture and kept at a regulated temperature of 37° C. using a heating pad. Aseptic surgery was performed to implant a cranial window providing optical access to cortical microvessels. In brief, a craniotomy (5×5 mm) was performed over the somato-sensory cortex and buffered with artificial cerebral spinal fluid. The craniotomy was sealed with a 150 μm cover glass and dental cement over the surrounding skull edge. The craniotomy protocol employed in studies reported here is commonly employed in cerebral blood flow studies and allows routine optical imaging of the murine brain while retaining physiological control (Drew et al., 2010). Different SaO₂ levels (51%, 57%, 71.5%, 80.6%, 92% and 99%) were achieved by varying the oxygen flow rate in a gas mixture composed of oxygen and nitrogen oxide. The DWP-OCT fiber probe was placed on top of the cover glass and directed at a target microvessel and data collected over a 10 second time period. The measurement protocol was repeated 5 times for each SaO₂ level.

Before DWP-OCT measurements, systemic blood oxygenation was stabilized at a fixed SaO₂ level for at least 5 minutes within ±2% using a MouseOx system (STARR Life Science Corp., Oakmont, Pa.) with the probe attached to the hindpaw of the mouse.

Selecting Target Arteriole in the Murine Brain.

Real-time data display together with small DWP-OCT probe diameter (125 μm) was utilized to record data from a target arteriole. OCT intensity including intensity M-mode images (FIG. 7C) do not directly detect location of murine brain microvessels (Wang and An, 2009). The inventors developed a multistep protocol that allowed to first, point DWP-OCT probe at a target arteriole, second maximally overlap the arteriole with DWP-OCT beams and third verify amplitude of the DWP-OCT signal was sufficient to provide meaningful SaO₂ measurements. First the DWP-OCT probe was pointed at the arteriole under the guidance of the surgical microscope (OMS-75, Topcon Medical Systems Inc., Oakland, N.J.). Identification of the arterioles was conducted by ascertaining the correct direction of branching, which are predominantly opposite to the draining venules in this region, and by smaller arteriole diameters due to higher order branching. This region is supplied by third- to fourth-order branches of the middle cerebral artery (MCA), which branch from the temporal lobe of the brain towards the medial to supply the cortical layers and drain into the venules. Further confirmation was obtained by visual inspection of the color of the surface microvasculature during pure oxygen inhalation, where a lighter red is associated with oxygenated hemoglobin versus dark crimson of deoxygenated hemoglobin in venous blood. Since the common-path interferometer employed here had a limited working distance (3.8 mm) precise placement of the DWP-OCT probe on the target arteriole was greatly facilitated by a miniature aperture (bare fiber cladding) and transparency of the probe. Next, the DWP-OCT probe was moved to maximize real-time speckle contrast from the target arteriole. The last step of insuring maximal overlap between photothermal excitation/OCT probe beams and the target arteriole was maximizing the amplitude of op variation at the modulation frequency of the 800 nm photothermal excitation beam. After obtaining maximal overlap between the target arteriole and DWP-OCT probe beams, the probe was fixed for the remainder time period for data acquisition from the arteriole. Optical pathlength (op) variations at the modulation frequency of phothermal excitation beams was not observed when the DWP-OCT probe was directed onto a murine brain region free of blood vessels.

### IV. Other Methods Using Photothermal-OCT

A. Imaging

Certain embodiments can be used to acquire 2D and 3D maps of functional information from a target, e.g., microvasculature within the tissue. For example, mammalian cells need an oxygen supply for their survival. The human body has a delicately organized vascular network that supplies our cells with oxygen, other nutrients, and removes waist products. Not surprisingly, aberrations in vascular oxygen supply are implicated in 70 disorders and that number continues to grow (Carmeliet, 2005). The oxygen distribution from vascular hemoglobin to the parenchymal cells begins with oxygen diffusion first from arterioles with diameters less than 50 μm, and second from capillaries, with deoxygenated blood draining back to venules where the blood is redirected to the lungs for re-oxygenation.

The earliest abnormalities in oxygen exchange take place at the capillary level and these can often be discerned clinically from oxygen extraction and blood flow measurements in arterioles and venules. Information from individual capillaries is frequently too localized and thus diffuse and requires a great deal of processing and is not easily to interpret, while information from larger (>50 μm diameter) arteries and veins can be dominated by nearby healthy tissue which mask localized pathology. Conversely, information acquired from target arterioles and venules ranging in diameters from 10-50 μm can provide relatively localized and early signs of oxygen delivery and consumption abnormalities that are easier to convert to clinically meaningful conclusions.

In certain aspects, the 2D and 3D functional maps (SaO₂ levels) can be achieved but not limited by introducing the scanning optics (mirrors M1, M2 in the FIGS. 14 and 15). The scanning optics for 2D and 3D imaging can be implementing using, for example, galvo-mirrors (GVS002, Thorlabs, N.J.). The 2D and 3D scanning can be implemented in two-arms (FIG. 14) and common-path (FIG. 15) interferometers. The procedure of providing map of SaO₂ levels comprises one or more of the following steps: (a) Identification of the target microvessel from the B-scan; (b) Tracking the microvessel during the measurement procedure; (c) Verification of the validity of the measurement; (d) Identification of another microvessel etc. The measurement procedure (steps a-c) should be fast to avoid influence of the motion artifacts and make the procedure comfortable for the patient. For example, the measuring time below 30 ms will avoid 90% of the motion artifact (Wyatt, 1968). The minimal achievable time for the measurement (τm) is ultimately limited by the time span between two neighbor A-scans of the OCT system (τA). Most time consuming step is identification of the target microvessel, where at least 1000 A-scans are needed and therefore τma=1000*τA. For example, the photothermal excitation time can be ultimately reduced to τmb=(Nm*Np)*τA≈100*τA where Nm≈10 is the number of A-scans per period of photothermal excitation light, Np≈10 number of photothermal excitation light periods per measuring time. Verification of the measurement validity and switching between tasks is also may take ˜10% of the τma. Therefore the inventors estimate τm=1200*τA per one SaO₂ level measurement. Suggesting average distance between microvessels of 200 μm (oxygen diffusion distance in tissues), parallel orientation of the microvessels along the flat tissue surface, 10 μm resolution along the blood vessel (FIG. 16) and commercially available τA=5 μs during comfortable time of τ=5 s for the patient an area of 1.3×1.3 mm can be investigated, where d=1.3 mm is a linear size of the area in both directions. The value d is estimated from the following equation:

τ=(N_{bx}*N_{by})τₘ, (28)

N_{bx}=d0.2 is a number of SaO₂ measurements in x-direction with the step of 0.2 mm, N_{by}=d0.01 is a number of SaO₂ measurements in y-direction with the step of 0.01 mm.

Maximal achieved τA=0.2 μs for OCT systems (Klein et al., 2011) allows to investigate 7.5×7.5 mm area during 5 s. Theoretically the time span between neighbor A-scans is limited by the bandwidth of the optical source (Δf=1/τAt) τAt=λ2/(c*Δλ)=3.6*10−14 s allowing to investigate area of 56×56 m during 5 s in theory. Here λ=1060 nm is a central wavelength of the source, Δλ=0.1 nm is a wavelength bandwidth of the source, and c=3*108 ms is the speed of light in vacuum.

Identification and Tracking of the Target Microvessel from the B-Scan.

The algorithm for the identification and tracking is presented in FIG. 17. Initial location of several microvessels is identified from B-scan using Doppler (Wang et al., 2008; Wang et al., 2009) or Speckle Contrast (SC) functionality (FIG. 18). The size (SC or Doppler), velocity (Doppler) and flow directions (Doppler) is calculated for all microvessels located in a B-scans with diameters ranging between 10 μm and 50 μm or as specified. Microvessels with positive and negative Doppler shift are separated and those with higher velocities or velocity to diameter ratios taken as arterioles. Next, the OCT beam is moved to the target microvessel to measure SaO₂ level. Automatic selection of the microvessel will be based on minimum deviation of the size and blood velocity to pre-specified values, which are different for arterioles and venules. If the software identifies and computes SaO₂ levels for multiple microvessels, previously investigated microvessels may be excluded from the list or measured multiple times for averaging. Manual selection of the microvessel for SaO₂ measurement from a real-time Doppler image can also be provided. To maintain photothermal excitation and DWP-OCT beams on the microvessel during time period required for SaO₂ measurements, Speckle Contrast (SC) feedback can be used (FIG. 17). Specifically, the position of the beams will be adjusted if standard deviation (SD) calculated from 5-10 intensity A-scans is lower than a specified value. SC has higher sensitivity and a shorter processing time than Doppler functionality, but Doppler functionality can also be used for the tracking purposes. Other tracking approaches based on motion tracking can be also used.

Verification of the Validity of the Measurement.

The important feature of the quantitative imaging device providing maps of microvascular blood oxygenation is to self-test measured values for consistency. In DWP-OCT the SaO₂ values are computed from normalized ratio of op changes due to phothermal excitation at two distinct wavelengths. OP changes at one wavelength (770 nm in preliminary studies) are sensitive to SaO₂ levels and op changes at 800 nm that is close to isobestic wavelength are insensitive to the SaO₂ levels. Isobestic wavelength is characterized by equal absorption of oxygenated and deoxygenated hemoglobin. The simultaneous use of three or more wavelengths (two or more sensitive and one insensitive to SaO₂ levels) in multiwavelength photothermal (MWP) OCT allows two or more independently computed SaO₂ levels at once from the same microvessel. If the difference between SaO₂ levels computed from, for example, op760/op800 and op780/op800 is below 4% the SaO₂ level will be calculated as an average of these two, otherwise the measurement will be repeated. Here op760, op780, op800—is an optical pathlength variations induced by phothermal excitation light at 760 nm, 780 nm and 800 nm correspondingly.

B. Combining SaO₂ and Actual Blood Flow Measurements.

Tissue oxygen supply and consumption requires combined measurement of the hemoglobin oxygen saturation (SaO₂) and actual blood flow (volumes). This can be done by combining suggested multi wavelength phothermal (MWP) OCT with pattern scanning Doppler OCT (Wang et al., 2008; Wang et al., 2009). In the pattern scanning Doppler OCT like in double-circle Doppler OCT (Wang et al., 2008; Wang et al., 2009) the angle between the directions of OCT probe beam and blood flow (needed for calculation of the actual blood flow velocity) is calculated from two crossings points between OCT probe beam and target blood vessel. Here the SaO₂ measurement can be supplement with actual blood flow velocity measurement. Actual blood flow velocity (ABFV) measurements can be performed using the same OCT system following the SaO₂ measurement in the same spatial points. Blood flow can be calculated from the product of blood flow velocity by blood vessel lumen cross-sectional area. Blood vessel lumen cross-sectional area can be measured using two or three dimensional Speckle Contrast or Doppler OCT images.

The procedure of providing a map of SaO₂ levels and ABFV consist of: Identification of the target microvessel from the B-scan as in SaO₂ measurements; SaO₂ measurement and verification as described above; OCT probe light pattern scanning to provide two or more crossing points between OCT probe light direction and target blood vessel; Calculation of the actual blood flow velocity; Verification of the validity of the actual blood flow velocity measurement; Identification of another microvessel etc.

Here pattern scanning of OCT probe light may need 2,000-3,000 A-scans, while other steps can be done much faster within few A-scans time spans. Therefore the time of the combined measurements of SaO₂ levels and ABFV may require 4-times longer than SaO₂ levels alone.

Verification of the validity of the actual blood flow velocity measurements can be performed in analog to the validity verification of the SaO₂ levels where OCT probe light pattern scanning provide more than two crossing points between OCT probe light direction and target blood vessel so the ABFV calculated from a pairs of crossing points can be compared to each other. The large variation between calculated ABFV from different crossing point pairs indicate the inconsistency in the measurements and measurements need to be repeated.

C. Oxygen Consumption Measurements

Another aspect is to separate arteries from veins for oxygen extraction calculations. Difference in the oxygenation levels between blood vessels from arterial and vein sides referred as oxygen extraction and show the relative amount of the oxygen extracted by tissues. More over the measuring of combined SaO₂ levels and ABFV provide information on actual amount of oxygen extraction in the tissues. To this end the inventor provide the following approach:

Relative oxygen extraction may be calculated as an average difference in SaO₂ levels between target arterioles and venules. Actual oxygen extraction may be calculated as (ABFV*SaO₂)_{A}−(ABFV*SaO₂)_{V} where sub A indicate arterial and sub V vein sites. To separate venules (veins, vein capillaries) and arterioles (arteries, arterial capillaries) the following procedure will be performed: Microvessels with positive and negative Doppler shift will be separated and those with higher velocities or velocity to diameter ratios taken as arterioles. Relative and actual oxygen extraction will be computed as an average difference in SaO₂ levels between specified number (for example, five) of target arterioles and venules that are in close proximity to each other.

To estimate SaO₂-OCT depth resolution the inventors approximate depth dependence of op (FIG. 17B) by assuming the photothermal temperature increase is a Guassian so that op varies as an error function: op∝Erf(z)=∫₀^{z}e^{-a}^{²}^{( z̅−z}^{₀}⁾^{²}d z̅, where z₀=568 μm is the center of absorption (arteriole) and a=0.031 μm⁻¹ is the parameter inversely proportional to the longitudinal size of the temperature increase. The longitudinal full width at the half maximum (FWHM) of the temperature increase z*=2\sqrt{ln}/a=45 μm gives SaO₂-OCT depth resolution to measure SaO₂ levels.

After target microvessel SaO₂ level and flow measurements, the nearest microvessel with opposite flow direction is detected using 400 μm radius (0.63 μm A-scan step) Doppler scan. Next, the SaO₂ level and flow for the nearest microvessel are calculated. From this pair the microvessel with higher blood velocity to diameter ratio is taken as the arteriole and the other as venule. The process is repeatedly applied to build a map of oxygen extraction values: O_{2E}=SaO_{2A}−SaO_{2V}, where SaO_{2A} and SaO_{2V} are the SaO₂ levels of arteriole and venule calculated for each arteriole/venule pair respectively. The relative oxygen consumption for the arteriole/venule pair is calculated as: O_{2C}=v_{A}S_{A}SaO_{2A}v_{V}S_{V}SaO_{2V}, where V_{A} and v_{V} are the blood flow velocities and S_{A} and S_{V} are the cross-sectional areas of the microvessel determined from Doppler OCT B-scans. The subscript A denotes arteriole and V denotes venule.

Consumption measurements can be used in assessing diabetic retinopathy. Diabetic retinopathy is the leading cause of blindness among the working age population. Degradation in autoregulation of the microvasculature oxygen extraction and vasodilation associated with blood flow has been implicated in the early stages of DR before anatomical changes can be detected. Autoregulation refers to a tissue's ability to adjust its blood flow and oxygen delivery in accordance with metabolic needs. Early detection of abnormal autoregulation profiles, when intervention is most effective, will dramatically improve DR treatment outcomes, DR progression prediction, and ultimately prevent blindness. In certain aspects of the invention, autoregulation can be assessed used the methods and apparatus described herein.

Existing non-invasive modalities to detect abnormal autoregulation are not layer-specific and are unable to differentiate information from the independently regulated retina and choroid and between two major retinal capillary layers. Optical Coherence Tomography (OCT) is a powerful clinical tool routinely used to non-invasively image retinal anatomy with depth-resolved layer information. Doppler OCT was proposed to study layer-specific retinal blood flow; however, non-invasive depth-resolved tissue oxygenation imaging has not been validated.

The examples as well as the figures are included to demonstrate certain embodiments of the invention. It should be appreciated by those of skill in the art that the techniques disclosed in the examples or figures represent techniques discovered by the inventors to function well in the practice of the invention, and thus can be considered to constitute modes for its practice. However, those of skill in the art should, in light of the present disclosure, appreciate that changes can be made in the specific embodiments disclosed and still obtain a like or similar result without departing from the spirit and scope of the invention.

### REFERENCES

The following references, to the extent that they provide exemplary procedural or other details supplementary to those set forth herein, are specifically incorporated herein by reference.
Adler et al., Optics Express 16, 4376-4393 (2008).
Adler et al., Optics Letters 32, 626-628 (2007).
Barton and Stromski, Opt. Express 13(14), 5234-5239 (2005).
Carmeliet and Jain, Nature 407, 249-257 (2000).
Chen et al. in Coherence Domain Optical Methods in Biomedical Science and Clinical Applications, Proceedings Of, V. V. Tuchin, H. Podbielska, B. Ovryn, and A. Katzir, eds. (1997), pp. 112-118.
Cheng et al., Proceedings of the National Academy of Sciences of the United States of America 103, 17525-17530 (2006).
Choma et al., J. Biomed. Opt. 10(4), 044009 (2005).
Choma et al., Optics Letters 30, 1162-1164 (2005).
Craft and Moe, Journal of Animal Science 12, 127-131 (1934).
Cutnell, Physics, 4th Edition ed. (John Wiley & Sons Inc., New York, 1997).
Dalziel, Biochemistry Journal 67, 119-124 (1957).
Drew et al., Nat. Methods 7(12), 981-984 (2010).
Dunn et al., Optics Letters 28, 28-30 (2003).
Faber et al., Optics Letters 28, 1436-1438 (2003).
Faber et al., Optics Letters 30, 1015-1017 (2005).
Grap, Critical Care Nurse 22, 8 (2002).
Grinvald et al., Nature 324, 361-364 (1986).
Izhaky et al., Japanese Journal of Ophthalmology 53, 345-351 (2009).
Jobsis, Science 198, 1264-1267 (1977).
Kagemann et al., Journal of Biomedical Optics 12 (2007).
Kamat, Indian Journal of Anaesthesia 46, 8 (2002).
Kuranov et al., IEEE Photon. Technol. Lett. 22(20), 1524-1526 (2010).
Kuranov et al., Photonics Technology Letters, IEEE 22, 1524-1526 (2010).
Kuranov ety al., Biomed. Opt. Express 2(3), 491-504 (2011).
Leitgeb et al., Opt. Express 11(23), 3116-3121 (2003).
Leitgeb et al., Optics Letters 25, 820-822 (2000).
Lu et al., Optics Letters 33, 416-418 (2008).
Mariampillai et al., Opt. Lett. 33(13), 1530-1532 (2008).
Matcher et al., Analytical Biochemistry 227, 54-68 (1995).
Mythen, Current Opinion in Critical Care 12, 3 (2006).
Paranjape et al., Biomed. Opt. Express 1, 2-16 (2010).
Petley et al., British Journal of Anesthesia 84, 28-32 (2000).
Prahl, “Optical Absorption of Hemoglobin” (1999), available on the world wide web at ornlc.ogi.edu/spectra/hemoglobin/.
Reynolds et al., British Medical Bulletin 44, 1052-1075 (1988).
Robles et al., Biomed. Opt. Express 1, 310-317 (2010).
Robles et al., Optics Express 17, 6799-6812 (2009).
Sirotin and Das, Nature 457, 475-U476 (2009).
Skala et al., Nano Letters 8, 3461-3467 (2008).
Vovenko, Pflugers Arch. 437(4), 617-623 (1999).
Wang and An, Opt. Express 17(11), 8926-8940 (2009).
Welch and van Gernert, Optical-Thermal response of laser-irradiated tissue, Laser, Photonics, and Electro-Optics (Plenum Press, New York, 1995).
Yi and Li, Optics Letters 35, 2094-2096 (2010).
Yun et al., Opt. Express 11(22), 2953-2963 (2003).
Zhao et al., Optics Letters 25, 114-116 (2000).
Zhou et al., Optics Letters 35, 700-702 (2010).

1. A method for measuring concentration of a constituent in a target comprising:
(a) exposing a target having two or more constituents to a first excitation radiation at a first wavelength that is absorbed by at least a first constituent and a second excitation radiation at a second wavelength that is absorbed by at least a second constituent,
(b) measuring optical path length changes in the target resulting from exposure of the target to the first and second excitation radiation, and
(c) determining a difference between the changes in optical path length relative to the first and the second excitation radiation and determining the levels of the first constituent relative to the second constituent by evaluating the optical path length changes.

2. The method of claim 1, wherein the first excitation radiation is differentially absorbed by the first and second constituent.

3. The method of claim 1, wherein optical path length changes are determined at two different depths within the target.

4. The method of claim 1, wherein the target is exposed to the first and second excitation radiation at the same time.

5. The method of claim 1, wherein measuring optical path length changes is by phase sensitive optical coherence tomography.

6. The method of claim 1, wherein the target is a biological target.

7. The method of claim 6, wherein the biological target is a tissue, organ, or biological fluid.

8. The method of claim 7, wherein the target is in a subject.

9. The method of claim 7, wherein the biological target is the retina.

10. The method of claim 9, wherein the biological target is a blood vessel in the retina.

11. The method of claim 7, wherein the biological target is a portion of an organ or tissue.

12. (canceled)

13. The method of claim 11, wherein the biological target comprises hemoglobin.

14. The method of claim 13, wherein the first constituent is oxygenated hemoglobin.

15. The method of claim 13, wherein the second constituent is deoxygenated hemoglobin, carboxy hemoglobin, sulf-hemoglobin, or methemo hemoglobin.

16. The method of claim 13, wherein hemoglobin oxygen saturation (Sa02) is measured.

17. The method of claim 13, wherein the first excitation radiation has a wavelength of about 800 nm.

18. The method of claim 13, wherein the second excitation radiation has a wavelength of about 765 nm.

19.-20. (canceled)

21. An multi wavelength photothermal optical coherence tomography apparatus comprising:
(a) an excitation radiation source configured to produce excitation radiation at least two distinct excitation wavelengths,
(b) a phase sensitive optical coherence detector, and
(c) data processing unit configured to process data related to optical path length changes in a target exposed to an excitation radiation.

22.-24. (canceled)

25. A method for mapping the presence of a constituent in a target comprising:
(a) exposing a target having at least one constituent to a first excitation radiation at a first wavelength that is absorbed by at least a first constituent,
(b) measuring optical path length changes in the target resulting from exposure of the target to the first excitation radiation at a plurality of locations within and/or on the target, and
(c) mapping the changes in optical path length resulting from the absorbtion of the first excitation radiation at the plurality of locations within and/or on the target.

26.-31. (canceled)
